(19) United States
(12) Patent Application Publication
(10) Pub. No.: US 2004/0133359 A1
(43) Pub. Date: Jul. 8, 2004

(54) Method and system for evaluation of electrical conductivity of dna sequences

(76) Inventor: Porat Erhlich, Boston, MA (US)

Correspondence Address: Mark Friedman, Bill Polkinghorn, Discovery Dispatch, 9003 Florin Way, Upper Marlboro, MD, 20772, US

(21) Appl. No.: 10/312,259

(22) PCT Filed: Jun. 25, 2001
(86) PCT No.: PCT/US01/20192

(51) Int. Cl. G06F 19/00; G01N 33/48; G01N 33/50

(52) U.S. Cl. 702/20

(57) ABSTRACT

Methods and a system for evaluating the electrical conductivity of a defined DNA sequence are disclosed. The method comprises the step of calculating the degree of asymmetry of the defined DNA sequence. A system for determining a measure of electrical conductivity of a defined DNA sequence is disclosed which comprises a computer containing within a memory device within it, an algorithm capable of calculating the degree of asymmetry of the defined DNA sequence. A method for the evaluation of the electrical conductivity of a defined DNA sequence is disclosed which comprises the step of providing instructions on a computer readable medium for a calculation of the degree of asymmetry of the defined DNA sequence.

### FIELD AND BACKGROUND OF THE INVENTION

The present invention relates to the field of genomics, and more particularly, to a method and system for evaluating the electrical conductivity of regions of DNA. The well known Watson & Crick model of the double helix describes the DNA molecule essentially as an elongated stack of aromatic nitrogenous base pairs wrapped with a ribbon of a negatively charged sugar phosphate polymer pair. Carbon atoms in the ring structures of the base pairs occur in SP2 hybridization and have π orbitals perpendicular to the ring planes. The intimate packing of adjacent base pairs in the core stack suggests a high degree of overlap between their π electron clouds. Since shortly after the Watson & Crick model was first proposed (Watson J D and Crick F H C (1953) Nature 171:737), scientists have begun entertaining the idea that the structure of the DNA molecule suggests that it may possess electric conductivity (Burnel et al. (1969) Ann N Y Acad Sci 158: 191-209; Holmlin R E (1997) Angew Chem Int Ed Engl 36, 2714-2730). This idea has been extensively developed and tested in recent years, both experimentally (Aich P et al. (1999) J Mol Biol 294: 477-85; al-Kazwini AT et. al. (11994) Radiat Res 138: 307-11; Arkin M R et al. (1996) Science 273: 475-80; Arkin M R et al. (1997) Chem Biol 4: 389400; Beach C et al. (1994) Radiat Res 137: 385-93; Dandliker P J et al. (1997) Science 275: 1465-8; Dandliker P J et al. (1998) Biochemistry 37: 6491-502; Das A et al. (1999) Chem Biol 6: 461-71; Debije M G and Bernhard W A (1999) Radiat Res 152: 583-589; Debije M G et al. (1999) Angew Chem Int Ed Engl 38: 2752-2756; Douki T and Cadet J (1999) Int J Radiat Biol 75: 571-81; Femandez-Saiz M et al. (1999) Photochem Photobiol 70: 847-52; Fink H W and Schonenberger C (1999) Nature 398: 407-10; Fuciarelli A F et al. (1994) Int J Radiat Biol 65: 409-18; Gasper S M (1997) J Am Chem Soc 119: 12762-12771; Hall D B (1997) J Am Chem Soc 119: 5045-5046; Hall D B et al. (1996) Nature 382: 731-5; Hall D B et al. (1998) Biochemistry 37: 15933-40; Henderson P T et al. (1999) Proc Natl Acad Sci USA 96: 8353-8; Holmlin RE (1997) Angew Chem Int Ed Engl 36: 2714-2730; Hyun K M et al. (1997) Biochim Biophys Acta 1334: 312-6; Jovanovic S V and Simic M G (1989) Biochim Biophys Acta 1008: 39-44; Kelley S O and Barton J K (1998) Chem Biol 5: 413-25; Kelley S O and Barton J K (1999) Science 283: 375-81; Kelley S O et al. (1999 Nucleic Acids Res 27: 4830-4837; Kielkopf C L et al. (2000) Nat Struct Biol 7: 117-21; Lewis F D et al. (1997) Science 277: 673-6; Meggers E et al. (2000) Chemistry 6: 485-92; Meggers E. (1998) J Am Chem Soc 120: 12951-12955; Murphy C J et al. (1994) Proc Natl Acad Sci USA 91: 5315-9; Murphy C J et al. (1993) Science 262: 1025-9; Napier M E et al. (1997) Bioconjug Chem 8: 906-13; Ninomiya K et al. (1999) Nucleic Acids Symp Ser 255-6; Nunez M E and Barton J K (2000) Curr Opin Chem Biol 4: 199-206; Nunez M E et al. (1999) Chem Biol 6: 85-97; Nunez M E et al. (2000) Biochemistry 39: 6190-6199; Ozaki H and McLaughlin LW (1992) Nucleic Acids Symp Ser 67-8; Porath D et al. (2000) Nature 403: 635-8; Ratner M (1999) Nature 397: 480-1; Razskazovskiy Y et al. (2000) Radiat Res 153: 43641; Ropp P A and Thorp H H (1999) Chem Biol 6: 599-605; Schuster GB (2000) Acc Chem Res 33: 253-260; Thompson M and Woodbury N W (2000) Biochermistry 39: 43274338; Wagenknecht H A et al. (2000) Biochemistry 39: 5483-5491; Wan C et al. (1999) Proc Natl Acad Sci USA 96: 6014-9; Wolf P et al. (1993) Int J Radiat Biol 64: 7-18; Xu D G and Nordlund T M (2000) Biophys J 78: 1042-58) and theoretically (Bixon M et al. (1999) Proc Natl Acad Sci USA 96: 11713-6; Buhks E and Jortner J (1980) FEBS Lett 109:117-20; Chojnacki H and Laskowski Z (1985) J Biomol Struct Dyn 2:759-65; Conwell E M and Rakhmanova SV (2000) Proc Natl Acad Sci USA 97:4556-60; Fonseca Guerra C and Bickelhaupt F M (1999) Angew Chem Int Ed Engl 38:2942-5; Grinstaff MW (1999) Angew Chem Int Ed Engl 38:3629-35; Jortner J et al. (1998) Proc Natl Acad Sci USA 95:12759-65; Meade T J (1996) Met Ions Biol Syst 32:453-78; Nunez M E and Barton J K (2000) Curr Opin Chem Biol 4:199-206; Onfelt B et al. (2000) Proc Natl Acad Sci USA 97: 5708-13; Schuster G B (2000) Acc Chem Res 33:253-60; Steenken S (1997) Biol Chem 378:1293-7; Steenken S (1992) Free Radic Res Commun 16:349-79; Takeda K (1995) Math Biosci 130:183-202; Wan C et al. (1999) Proc Natl Acad Sci USA 96:6014-9.)

Recent work carried out in a number of independent laboratories, using various biophysical methodological approaches, demonstrated that long-range migration of charge through the double helix occurs (Aich P et al. (1999) J Mol Biol 294: 477-85; al-Kazwini A T et. al. (1994) Radiat Res 138: 307-11; Arkin MR et al. (1996) Science 273: 475-80; Arkin M R et al. (1997) Chem Biol 4: 389-400; Beach C et al. (1994) Radiat Res 137: 385-93; Clery D (1995) Science 267:1270; Dandliker P J et al. (1997) Science 275: 1465-8; Dandliker P J et al. (1998) Biochemistry 37: 6491-502; Das A et al. (1999) Chem Biol 6: 461-71; Dunn DA et al., (1992) Biochemistry 31: 11620-5; Fink H W and Schonenberger C (1999) Nature 398: 407-10; Hall D B et al. (1996) Nature 382: 731-5; Hall D B et al; (1998) Biochemistry 37: 1593340; Henderson P T et al. (1999) Proc Natl Acad Sci USA 96: 8353-8; Holmlin R E (1997) Angew Chem Int Ed EngI 36: 2714-2730; Jovanovic S V and Simic M G (1989) Biochirn Biophys Acta 1008: 3944; Murphy CJ et al. (1994) Proc Natl Acad Sci USA 91: 5315-9; Murphy C J et al. (1993) Science 262: 1025-9; Nunez M E and Barton J K (2000) Curr Opin Chem Biol 4: 199-206; Nunez M E et al. (2000) Biochemistry 39: 6190-6199; Porath D et al. (2000) Nature 403: 635-8; Wagenknecht H A et al. (2000) Biochemistry 39: 5483-5491; Wan C et al. (1999) Proc Natl Acad Sci USA 96: 6014-9) and that it is sequence and π stacking sensitive (Arkin M R et al. (1996) Science 273: 475-80; Fuciarelli A F et al. (1994) Int J Radiat Biol 65: 409-18; Hall D B (1997) J Am Chem Soc 119: 5045-5046; Kelley S O and Barton J K (1998) Chem Biol 5: 413-25; Kelley S O and Barton J K (1999) Science 283: 375-81; Meggers E et al. (2000) Chemistry 6: 485-92; Meggers E. (1998) J Am Chem Soc 120: 12951-12955; Napier M E et al. (1997) Bioconjug Chem 8: 906-13; Nunez M E and Barton J K (2000) Curr Opin Chem Biol 4: 199-206; Nunez M E et al. (1999) Chem Biol 6: 85-97; Ropp P A and Thorp H H (1999) Chem Biol 6: 599-605; Schuster GB (2000) Acc Chem Res 33: 253-260; X U D G and Nordlund T M (2000) Biophys J 78: 1042-58.) While most of the direct evidence supporting DNA charge migration was obtained in highly artificial experimental systems, the impact of these findings on biological research may be immense because they raise the possibility that the double helix may have a capacity to conduct charge also under normal physiological conditions (Blank M and Goodman R (1997) Bioelectromagnetics 18:111-5; Blank M and Goodman R (1999) J Cell Biochem 75: 369-74; Buhks E and Jortner J (1980) FEBS Lett 109:117-20; Cullis P M et. al. (1987) Nature 330:7734.)

DNA is the universal carrier of genetic information. If it is true that charge transfer through the double helix exists under normal physiological conditions in cells of living organisms, it could have a profound impact on the understanding of the function of genetic material and processes which take place directly on it (such as transcription and replication). It would not be impossible that charge transfer in the double helix is utilized by the cell as a component of the control mechanisms that govern gene expression.

In light of the results demonstrating DNA charge migration in vitro, prominent researchers in the field have expressed the opinion that attempts should be directed at studying the biological implications of the phenomenon (Blank M and Goodman R (1997) Bioelectromagnetics 18:111-5; Blank M and Goodman R (1999) J Cell Biochem 75: 369-74; Grinstaff M W (1999) Angew Chem Int Ed Engl 38:3629-35; Holmlin R E (1997) Angew Chem Int Ed Engl 36: 2714-2730; Lin H et. al. (1998) J Cell Biochem 70: 297-303; Nunez M E and Barton J K (2000) Curr Opin Chem Biol 4: 199-206; Nunez M E et al. (1999) Chem Biol 6: 85-97.) If charge migration serves a biological function, then traces of the phenomenon may be predicted to have been registered into the genome by evolution. To date, there has been no publication of any experimental approach for rigorous testing of hypotheses on the putative biological effects of DNA charge migration, nor has there been any publication of a method for determining the electrical conductivity properties of a selected DNA segment.

Biophysical evidence (see references hereinabove) suggests that charge migration is sequence sensitive. If this is true then the charge conductivity qualities of a given region in a chromosome are dictated by the sequence of bases. If a particular region in genomic DNA functions as a modulator of charge conductivity, its structure can be expected to reflect that fact. Non-coding regions of genomic DNA do not code for proteins but they still code for their own physicochemical characteristics. In DNA ‘structure=sequence’ and structural characteristics of the molecule can therefore be deduced from sequence analysis. Thus, depending on its specific sequence, the electrical conductivity properties of a region of DNA might be predicted. There is no prior art suggesting that, or how, this might be accomplished while prior art methods and systems for analysis of DNA sequence exist, there are no prior art systems that provide a method or means for analysis of a DNA sequence for determination of its electrical conductivity properties.

For example, prior art systems (Brendel V and Trifonov E N (1984) Nucleic Acids Res 12:4411-27; Cox R and Mirkin S M (1997) Proc Natl Acad Sci USA 94:5237-42; Day G R and Blake R D (1982) Nucleic Acids Res 10:8323-39; Karlin S (1986) Proc Natl Acad Sci USA 83:6915-9; Karlin S and Brendel V (1992) Science 257:3949; Karlin S et. al. (1992) Nucleic Acids Res 20:1363-70; Karlin S and Cardon L R (1994) Annu Rev Microbiol 48:619-54; Karlin S and Ghandour G (1985) J Mol Evol 22:195-208; Karlin S et al. (1983) Proc Natl Acad Sci USA 80:56604; Karlin S et. al. (1988) Comput Appl Biosci 4:41-51; Karlin S et. al. (1988) Proc Natl Acad Sci USA 85:841-5; Korn L J et. al. (1977) Proc Natl Acad Sci USA 74:4401-5; Mount D W and Conrad B (1984) Nucleic Acids Res 12:811-7; Munroe S H (1983) Nucleic Acids Res 11:8891-900; Nussinov R (1991) DNA Seq 2:69-79; Pivek et.al. (1985) Folia Biol (Praha) 31:213-34; Schroth GP and Ho P S (1995) Nucleic Acids Res 23:1977-83) have been described which utilize algorithms to analyze DNA sequences for restriction sites, homologies to other sequences, or homologous elements such as repetitive elements and complete dyad symmetries and palindromes, in order to compare genomes of different species, and to identify potential cruciform and hairpin loop secondary structures. These various computer algorithms, based on different methods of operation, have been devised to locate dyad symmetrical elements in sequence and determine their frequency and distribution in order to predict secondary structure of the nucleic acid molecules. However, all these systems suffer from limitations, such as that they are designed to identify symmetry and not asymmetry (generally functioning by attempting to locate two complementary and reversed occurrences of the same nucleotide sequence in close proximity on the same strand.) They were not designed to or able to quantitate the degree of asymmetry, even though some are tolerant to minor degrees of mismatch. None provides a means for analysis of a DNA sequence for determination of its electrical conductivity properties.

Most of the work on functional characterization of genomic DNA sequence (locating functional elements such as promoters, enhancers, splice sites, and origins of replication, for example) is empirical. Most of the data in the field is obtained through elaborate and time consuming in-vivo and in vitro assays. A need exists for computer based tools, which enable scientists to accurately predict functional elements in DNA sequence by means of sequence analysis. Tools of this kind can is reduce the amount of costly and time consuming ‘wet’ experiments, and enhance the efficiency of the process of understanding the complexity of the genome. Effective tools for assembling short fragments of expressed sequence into putative exons are in widespread commercial and academic use, providing an example of the need and demand for such tools in general. A particular demand exists for new and improved tools that would enable prediction of the location and strength of elements involved in gene expression, such as promoters and enhancers with enhanced accuracy and precision. Such improved tools could reduce the amount of labor required by current methodologies for determination of the location of transcription start sites and for evaluation of the strength of promoters and enhancers. It could also be used for the study and diagnosis of various genetic diseases and conditions, where these are caused by mutations in the regulatory elements of genes rather than by mutations in protein coding sequences.

Information written in linear form can be expected to possess directionality. For a sequence of characters dedicated to storing information, the more unique and specific the information, the less sense it makes read backwards. The double helix is, indeed, dedicated to storing genetic information, but it is also dedicated to self-propagation. These two separate functions maintained by DNA, namely storing genetic information as well as perpetuating it, impose two separate sets of structural constraints on the molecule. The former is manifested in the molecule having a varying sequence of bases, containing the information, while the latter dictates the double stranded structure with its rigid base pairing rules. In order for the information carried in DNA to be both unique and legible to the executing apparatus, it needs to be unidirectional. In order for it to be efficiently replicated and passed on to progeny, it needs to have an anti-parallel counterpart. In the majority of information-containing regions in genomic DNA, the sequence of one of the strands can therefore be expected to store the unique information, while the sequence of the other is a nonsense, reverse complement version of that information.

Without any prior knowledge of what the information might be, or what the coding rules for that information are, it is difficult to tell which of the strands encodes the (sense) information (and which is simply the anti-sense complement) because there is no physical difference between them, except in their base sequence and either one could do the job equally well.

This genetic information is packed and stored in DNA, in systems of molecular information storage and executed by molecular execution mechanisms. One outstanding example of such a system of molecular information storage is the ‘Genetic Code’, which provides the set of transition rules from DNA sequence to protein sequence, and the ribosome which executes the transfer of genetic information by synthesizing polypeptides in the process of protein translation. But as >95% of the total quantity of genomic DNA in humans does not contain protein code, it is widely believed that other less well understood forms of information storage exist in the genome. One example of such a hypothesized but not yet fully elucidated system of information storage is the set of instructions which directs the process of transcription. This system probably includes specific transcription factor binding elements as well as other codes and forms of information storage.

The common denominator between systems of DNA information storage is that it is likely that in all of them the method of storage is based on unique permutations in the order of bases the sequence. Therefore there exists an acute need for a method and a system, with a general applicability to nucleic acid systems of information storage, that will detect and measure the information content along nucleic acid sequence and also assist in decoding that information, without prior knowledge of the execution apparatus. It would further be desirable to have a method and system for identifying regions of DNA that convey specific information content, such as protein coding sequence.

Thus there is an unmet need for, and it would be highly advantageous to have, a method for identifying DNA sequences that are good candidates to effectively propagate charge transfer and serve as an electrical conductor. There is further an unmet need for, and it would be highly advantageous to have, computer based tools to enable scientists to accurately predict functional elements, such as transcription related functional elements, in DNA sequence by means of sequence analysis. Further, there is an unmet need for a system and method that can identify regions of DNA or RNA which contain protein-coding information. Further, there is an unmet need for a system and method that can identify regions in nucleic acid sequences, which contain any form of coded genetic information in general.

### SUMMARY OF THE INVENTION

According to the present invention there is provided methods and a system to determine the electrical conductivity properties of a DNA sequence. Further, there is provided a method and a system for identifying functional elements in a DNA sequence. Still further there is provided a method and a system for predicting protein-coding information content in a nucleic acid sequence. Thus the present invention provides methods and systems for identifying information containing regions in general along a nucleic acid sequence and assisting in decoding that information.

According to one aspect of the present invention there is provided a method for determining a measure of electrical conductivity of a defined DNA sequence, which comprises the step of calculating the degree of asymmetry of the defined DNA sequence.

According to another aspect of the present invention there is provided a system for determining a measure of electrical conductivity of a defined DNA sequence, which comprises a computer containing within a memory device thereof, an algorithm which is capable of calculating the degree of asymmetry of the defined DNA sequence.

According to yet another aspect of the present invention there is provided a method for the evaluation of electrical conductivity of a defined DNA sequence which comprises the step of providing instructions on a computer readable medium for a calculation of the degree of asymmetry of the defined DNA sequence.

According to still another aspect of the present invention there is provided a method for identifying functional elements in a DNA sequence, the method including the steps of: (a) calculating at least one set of dyad pair type frequencies within a portion of the DNA sequence equal in size to two times a window size, around at least one potential axis of dyad symmetry in the DNA sequence, and (b)

based on the at least one set of dyad pair type frequencies, identifying regions of the DNA sequence containing the functional elements.

According to an additional aspect of the present invention there is provided a method of identifying transcription related functional elements, including the steps of: (a) calculating at least one set of dyad pair type frequencies within a portion of the DNA sequence equal in size to two times a window size, around at least one potential axis of dyad symmetry in the DNA sequence, and (b) based on the at least one set of dyad pair type frequencies, identifying regions of the DNA sequence containing the functional elements.

According to yet an additional aspect of the present invention there is provided a system for identifying functional elements in a DNA sequence, the system including: (a) a software module including a plurality of instructions for calculating at least one set of dyad pair type frequencies within a portion of the DNA sequence equal in size to two times a window size, around at least one potential axis of dyad symmetry in the DNA sequence, (b) a memory for storing the instructions, and, (c) a processor for executing the instructions.

According to still an additional aspect of the present invention there is provided a computer readable storage medium having computer readable code embodied on the computer readable storage medium, the computer readable code for identifying functional elements in a DNA sequence, the computer readable code comprising: program code including a plurality of instructions for calculating at least one set of dyad pair type frequencies within a portion of the DNA sequence equal in size to two times a window size, around at least one potential axis of dyad symmetry in the DNA sequence

According to still an additional aspect of the present invention there is provided a method for determining electrical conductivity properties of a DNA sequence, the method comprising the steps of: (a) calculating at least one set of dyad pair type frequencies within a portion of the DNA sequence equal in size to two times a window size, around at least one potential axis of dyad syrnmetry in the DNA sequence, and, (b) based on the at least one set of dyad pair type frequencies, determining the electrical conductivity properties of the DNA sequence.

According to still an additional aspect of the present invention there is provided a system for determining electrical conductivity properties of a DNA sequence, the system including: (a) a software module including a plurality of instructions for calculating at least one set of dyad pair type frequencies within a portion of the DNA sequence equal in size to two times a window size, around at least one potential axis of dyad symmetry in the DNA sequence, (b) a memory for storing the instructions, and, (c) a processor for executing the instructions.

According to yet an additional aspect of the present invention there is provided a computer readable storage medium having computer readable code embodied on the computer readable storage medium, the computer readable code for determining electrical conductivity properties of a DNA sequence, the computer readable code comprising: program code including a plurality of instructions for calculating at least one set of dyad pair type frequencies within a portion of the DNA sequence equal in size to two times a window size, around at least one potential axis of dyad symmetry in the DNA sequence.

According to still an additional aspect of the present invention there is provided a method for identifying protein coding regions in a nucleic acid sequence, the method comprising the steps of: (a) calculating at least one set of dyad pair type frequencies within a portion of the nucleic acid sequence equal in size to two times a window size, around at least one potential axis of dyad symmetry in the nucleic acid sequence, and (b) based on the at least one set of dyad pair type frequencies, identifying the protein coding regions contained within the nucleic acid sequence.

According to still an additional aspect of the present invention there is provided a system for identifying protein coding regions in a nucleic acid sequence, the system comprising: (a) a software module including a plurality of instructions for calculating at least one set of dyad pair type frequencies within a portion of the nucleic acid sequence equal in size to two times a window size, around at least one potential axis of dyad symmetry in the nucleic acid sequence, (b) a memory for storing the instructions, and, (c) a processor for executing the instructions.

According to still an additional aspect of the present invention there is provided a computer readable storage medium having computer readable code embodied on said computer readable storage medium, the computer readable code for identifying protein coding regions in a nucleic acid sequence, the computer readable code comprising: program code including a plurality of instructions for calculating at least one set of dyad pair type frequencies within a portion of the nucleic acid sequence equal in size to two times a window size, around at least one potential axis of dyad symmetry in the nucleic acid sequence.

According to still an additional aspect of the present invention there is provided a method for identifying information containing regions in a nucleic acid sequence, the method comprising the steps of: (a) calculating a degree of asymmetry of the nucleic acid sequence within a portion of the nucleic acid sequence, around at least one potential axis of dyad symmetry in the nucleic acid sequence, and (b) based on said degree of asymmetry, identifying the information containing regions contained within the nucleic acid sequence.

According to still an additional aspect of the present invention there is provided a system for identfying information containing regions in a nucleic acid sequence, the system comprising: (a) a software module including a plurality of instructions for calculating a degree of asymmetry of the nucleic acid sequence within a portion of the nucleic acid sequence, around at least one potential axis of dyad symmetry in the nucleic acid sequence, (b) a memory for storing the instructions, and, (c) a processor for executing the instructions.

According to still an additional aspect of the present invention there is provided a computer readable storage medium having computer readable code embodied on the computer readable storage medium, the computer readable code for identifying information containing regions in a nucleic acid sequence, the computer readable code comprising: program code including a plurality of instructions for calculating a degree of asymmetry of the nucleic acid sequence within a portion of the nucleic acid sequence, around at least one potential axis of dyad symmetry in the nucleic acid sequence.

According to further features in preferred embodiments of the invention described hereinbelow, the calculation of degree of asymmetry is accomplished by calculating at least one polarity value around at least one potential axis of dyad symmetry in the DNA sequence, where the polarity value is a unit-less number defined as (1−[S/W]), where S represents a number of dyad-symrnetrical bases and W represents a window size.

According to still further features in preferred embodiments of the present invention, the at least one polarity value is an ordered series of polarity values iteratively calculated for each potential axis in the DNA sequence.

According to still further features in preferred embodiments of the present invention, the series of polarity values is plotted graphically, whereby extended regions in the DNA sequence that possess values of polarity which deviate from expected polarity values of a random sequence may be identified.

According to still further features in preferred embodiments of the present invention, the series of polarity values is subjected to statistical analysis, whereby extended regions in the DNA sequence that possess values of polarity which deviate from expected polarity values of a random sequence may be identified.

According to still further features in the described preferred embodiments of the present invention, the DNA sequence length is in the range of 2 bases to 3×10⁹ bases.

According to still further features in preferred embodiments of the invention described below, the window size is an independent variable, with values ranging from 1 to a value equal to that of the largest whole integer smaller than one half the length of the DNA sequence, to be designated prior to calculation.

According to still further features in preferred embodiments of the invention described below, at least one of the at least one set of dyad pair type frequencies is an ordered array of the dyad pair frequencies.

According to still further features in preferred embodiments of the invention described below, the calculating of at least one set of dyad pair type frequencies is effected iteratively for each of the at least one potential axis of dyad symmetry in the DNA sequence.

According to still further features in preferred embodiments of the invention described below, the step of identifying regions of the DNA sequence containing the functional elements is effected by steps including subjecting the at least one set of dyad pair type frequencies to statistical analysis, whereby at least one region in the DNA sequence is identified that possesses at least one statistical value that indicates that an observed at least one set of dyad pair type frequencies deviates from an expected at least one set of dyad pair type frequencies.

According to still further features in preferred embodiments of the invention described below, the at least one statistical value is chosen from the group consisting of residuals of the dyad pair type frequencies, chi-square values, and likelihood ratios.

According to still further features in preferred embodiments of the invention described below, the statistical analysis includes plotting said statistical values.

According to still further features in preferred embodiments of the invention described below, the window size is an independent variable, having a value of at least 1 and at most one half a length of the DNA sequence.

According to still further features in preferred embodiments of the invention described below, the calculating of the at least one set of dyad pair type frequencies is performed on at least one dyad pair wherein both nucleotides of the at least one dyad pair are located on a single stand of the DNA sequence.

According to still further features in preferred embodiments of the invention described below, the calculating of the at least one set of dyad pair frequencies is performed on at least one dyad pair wherein both nucleotides of the at least one dyad pair are located on complementary strands of the DNA sequence.

According to still further features in the described preferred embodiments of the present invention, the step of identifying the protein coding regions contained within the nucleic acid sequence is effected by steps including comparing two of the sets of dyad pair type frequencies from around two adjacent potential axes of dyad symmetry in the nucleic acid sequence.

According to still further features in the described preferred embodiments of the present invention, comparing two of the sets of dyad pair type frequencies is effected by steps including calculating a sum of squares differences between the dyad pair type frequencies from around two adjacent potential axes of dyad symmetry in the nucleic acid sequence.

According to still further features in the described preferred embodiments of the present invention, one potential axis of dyad symmetry of the two adjacent potential axes of dyad symmetry in the nucleic acid sequence is located on a base of the nucleic acid sequence and the other potential axis of dyad symmetry is located off the base.

According to still further features in the described preferred embodiments of the present invention, comparing is effected iteratively for each potential axis of dyad symmetry in the nucleic acid sequence.

According to still further features in the described preferred embodiments of the present invention, calculating the dyad pair type frequencies is performed on at least one dyad pair where both nucleotides of the dyad pair are located on a single strand of the nucleic acid sequence.

According to still further features in the described preferred embodiments of the present invention, calculating the dyad pair type frequencies is performed on at least one dyad pair where both nucleotides of the dyad pair are located on complementary strands of the nucleic acid sequence.

According to still further features in the described preferred embodiments of the present invention, calculating the degree of asymmetry is effected by steps including calculating at least one set of dyad pair type frequencies within the portion of the nucleic acid, around at least one potential axis of dyad symmetry in the nucleic acid sequence.

According to still further features in the described preferred embodiments of the present invention, calculating the degree of asymmetry is effected iteratively for each potential axis of dyad symmetry in the nucleic acid sequence.

According to still further features in the described preferred embodiments of the present invention, the portion of the nucleic acid sequence is equal in size to two times a window size.

The method and system of the present invention can be used as a research instrument to enable the search for evidence supporting the theory of DNA charge migration in DNA sequence. If charge migration serves a biological function, then traces of the phenomenon may be registered into the base sequence by evolution. As will be shown below, examination of specific DNA sequences, such as the genome of the human immunodeficiency virus, HIV2, with this method and system has been accomplished yielding the unexpected result that several apparent extended regions of increased polarity are easily identified.

When applied to genomic DNA sequence of various organisms, particularly human, the method can locate distinct, recognizable elements in DNA. When the location of these distinct elements is superimposed on the functional map of genes, it is evident that a significant degree of overlap exists. One striking example is the element that is found to reside on the transcription initiation point of many human genes. The system and method of the present invention, by detecting this element, can accurately predict the location of promoters of genes. It appears that not only the location but also the strength of a promoter can be predicted. Some genetic diseases and conditions, such as Fragile-X syndrome, for example, are caused by mutations of the control regions of genes rather than of their protein coding regions. When affected and non-affected alleles of the FMR1 gene (known to be responsible for Fragile-X syndrome) are analyzed using the present invention, a clearly visible difference is found between the transcription initiation site elements of the affected and non-affected alleles. As many genetic conditions may be caused by mutations in control regions, the system and method of the present invention will therefore contribute to a better understanding of the control of gene expression in both normal and pathological situations.

The method and system of the present invention further has the potential to serve as a tool for predicting protein coding information content, based on sequence analysis alone, thus saving on expression library screening and other costly laboratory procedures. It may also prove useful in gene discovery as some RNA transcripts expressed in low abundance or in extremely narrow time windows in development, are virtually absent from expression libraries and can only be inferred from sequence. The present invention includes a method with a general applicability to nucleic acid systems of information storage, that will detect and measure the information content along a nucleic acid sequence and also assist in decoding that information. A preferred embodiment of such a method and a system for executing it is presented here which determines the level of dyad symmetry across potential axes of dyad symmetry in the nucleic acid molecule. The ability of this method to predict the electrical conductivity of regions in DNA, as part of a putative mechanism of information storage related to transcription control; an alternative preferred embodiment with the ability to predict functional elements of transcription; and another preferred embodiment with the ability to predict protein-coding regions demonstrate that information containing regions of nucleic acid may be identified by analysis of their sequence alone.

The present invention thus successfully addresses the shortcomings of the presently known configurations by providing a method and a system for the analysis of a defined nucleotide sequence to calculate the degree of asymmetry in that sequence in order to determine the electrical conductivity properties of a DNA sequence. The present invention further provides a method and a system for identifying functional elements within a DNA sequence. Still further there is provided a method and a system for predicting protein-coding information content in a nucleic acid sequence. Thus the present invention provides methods and systems for identifying information containing regions in general along a nucleic acid sequence and assisting in decoding that information.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a drawing that schematically illustrates a fragment of duplex DNA, 16 base pairs long (an example of an input sequence); along the top of the figure, potential axes of symmetry are indicated;

FIG. 1B is a flow diagram indicating the major steps in the analysis of the input sequence; for illustrative purposes only, the window size is set here to five nucleotides; dyad-synunetrical bases are indicated by bold typeface; each iterative step is representative of the application of the formula to calculate polarity at a different potential axis of symmetry; the input sequence and potential axes of symmetry are as indicated in FIG. 1A;

FIG. 1C is a table listing the polarity values for the 13 potential axes calculated in an example analysis of the input sequence in FIG. 1A, using a window size of five, according to steps as illustrated in FIG. 1B;

FIG. 1D is a graphic presentation of the output list of polarity values, taken from the example of FIG. 1C; the expected value of 0.75 is also indicated;

FIG. 2A is a graphic presentation of the output list of polarities from an analysis of the complete genome of HIV2 using an embodiment of the present invention; extended regions of increased polarity are indicated, these being regions of 500-600 nucleotides where values of polarity are concentrated which deviate from the 0.75 expected, and thus determined to be regions that would function effectively to propagate electron transfer and serve as an electrical conductor;

FIG. 2B is a graphic presentation of the output list of an analysis of a randomly generated mock DNA sequence illustrating no significant extended deviation from the expected 0.75 value.

FIG. 3 is a flow chart illustrating a preferred embodiment of the present invention;

FIG. 4 is a detailed flow chart of a computer algorithm further illustrating an example of a possible configuration of the present invention with iterative calculation of polarity values for multiple potential axes of symmetry for a DNA sequence;

FIG. 5A is a table illustrating the 16 dyad pair types;

FIG. 5B is a drawing that schematically illustrates a fragment of duplex DNA, 8 base pairs long (an example of an input sequence); a potential axis of symmetry at the center of the fragment and a window size of 4 are indicated;

FIG. 6 is a flow chart illustrating an alternate preferred embodiment of the present invention;

FIG. 7 is a detailed flow chart of a computer algorithm further illustrating an example of a possible configuration of a preferred embodiment of the present invention with iterative calculation of dyad pair type frequencies for multiple potential axes of symmetry for a DNA sequence;

FIG. 8 shows output plots of dyad pair type frequency analyses of nine genomic sequence fragments and a control fragment of computer generated random sequence;

FIG. 9 shows a dyad pair type frequency analysis of two DNA sequences, the FMR1 fragment in FIG. 9A and the “FMR1+(CGG)₃₃₃” fragment in FIG. 9B;

FIG. 10 is a high level block diagram of a system for predicting the electrical conductivity properties and for identifying functional elements in a defined DNA sequence according to the present invention;

FIG. 11 is a flow chart showing an alternate preferred embodiment of the present invention for assessing information content related to protein coding;

FIG. 12 (parts A, B, C, and D) is a detailed flow chart of a computer algorithm further illustrating an example of a possible configuration of a preferred embodiment of the present invention used for assessing information content related to protein coding; and,

FIG. 13 is the output from a dyad pair type frequency analysis of three human genes showing the identification of the protein-coding region of those genes.

### DETAILED DESCRIPTION OF THE INVENTION

The present invention is of a method and system consisting of a computer algorithm which can be used to determine a measure of electrical conductivity of a defined DNA sequence. Specifically, the present invention can be used to calculate the defined DNA sequence's degree of asymmetry over an extended length. Furthermore, the present invention is used to identify functional elements, in particular transcription-related functional elements, within the DNA sequence. Still further the present invention can be used to identify regions of nucleic acid sequence which contain protein-encoding information. Yet further, the present invention thus can be used to locate and decode information-containing regions of nucleic acid sequence.

The principles and operation of a method and system to determine a measure of electrical conductivity of a defined DNA sequence according to the present invention may be better understood with reference to the drawings and accompanying descriptions. For purposes of this specification and accompanying claims, the phrase, “dyad symmetry” is defined as the following: A DNA nucleotide sequence is said to show complete dyad symmetry when the base sequence at a particular position relative to an axis perpendicular to the DNA sequence on one strand of double-stranded DNA is identical to the base sequence on the complementary strand at a position equidistant from the axis, although in opposite orientation (that is, reading left to right on the upper strand for example, and right to left on the complementary lower strand). The degree to which the base sequence at a particular position relative to an axis perpendicular to the major longitudinal axis of DNA molecule on one strand of double-stranded DNA is identical to the base sequence on the complementary strand at a position equidistant from the axis indicates the degree of symmetry of that sequence if they are less than completely identical. Two bases are said to be dyad symmetric when the two bases, at the same position (distance) relative to an axis perpendicular to the major longitudinal axis of DNA molecule, but located on opposite strands of double stranded DNA, are identical. For purposes of this specification and accompanying claims, in certain configurations of the present invention two bases may also be considered dyad symetric (that is there is dyad symmetry present) when the two bases, at the same position (distance) relative to an axis perpendicular to the major longitudinal axis of DNA molecule, but located on opposite strands of double stranded DNA, are not identical, but both belong to the same family of bases, that is, both are either purines or pyrimidines.

For purposes of this specification and accompanying claims, the phrase, “axis of symmetry” is defined as an axis perpendicular to the major longitudinal axis of DNA molecule around which the nucleotide sequence can be analyzed to determine the degree to which the nucleotide sequence on one strand is identical to the base sequence on the complementary strand at a position equidistant from the axis, although in opposite orientation (that is, reading left to right on the upper strand for example, and right to left on the complementary lower strand). Because dyad symmetry may or may not be present around any given axis chosen, the axis may preferably be referred to as a potential axis of dyad symmetry. For the purposes of this specification and the accompanying claims, the terms “axis of symmetry”, “axis of dyad symmetry,” “potential axis of symmetry,” and “potential axis of dyad symmetry” shall be interpreted as meaning the same thing. For purposes of this specification and accompanying claims, the phrase, “window of symmetry” or “window size” is defined as the length in bases of the sequence being tested for identity at each side of any potential axis of symmetry.

Specifically envisioned as being within the scope of the present invention is the use with nucleic acid sequences including both DNA, and RNA of all types, including artificial and recombinant molecules as well as naturally occurring ones.

Before explaining at least one embodiment of the invention in detail, it is to be understood that the invention is not limited in its application to the details of construction and the arrangement of the components set forth in the following description or illustrated in the drawings (for example, any particular software programming language). The invention is capable of other embodiments or of being practiced or carried out in various ways. Also, it is to be understood that the phraseology and terminology employed herein is for the purpose of description and should not be regarded as limiting.

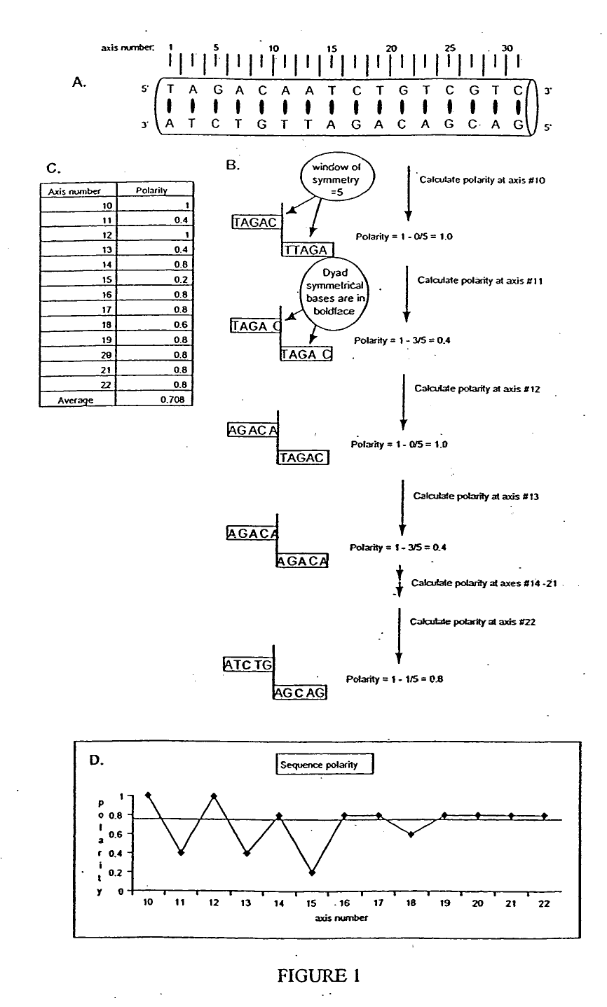

Any given fragment of double stranded DNA has two complementary 5′→3′ sequences, one for each strand. While in some cases (i.e., in perfect palindromes) these sequences may be identical, in the majority of circumstances they are different from each other (see FIG. 1A). Comparing the 5′→3′ sequence of a DNA fragment to the 5′→3′ sequence of the complementary strand of the same fragment is equivalent to comparing the two paths that a hypothetical test charge migrating through the fragment of DNA in either direction could take. Analogous to a diode, if a region of DNA has evolved to function as a charge conductivity modulation element, it is unlikely to exert its action in both directions equally and its sequence is therefore predicted to show a distinct directionality. Such directionality in a sequence can be revealed by a systematic analysis of polarity, that is, the extent of sequence asymmetry of the complementary strands over an extended length of base pairs. Regions of DNA with enhanced charge conductivity will be identified as extended regions with increased polarity as compared with expected. Extended regions with decreased polarity as compared to expected are also identified and are also predicted to possess unique charge conductivity properties, namely high resistance.

The input to the algorithm is a string of characters representing the order of nucleotide bases from a single strand of a molecule of DNA. Depending on the specific embodiment of the present invention, the output is a number or a series of numbers each representing the polarity value of one potential axis of dyad symmetry in the input sequence. Polarity is a unit-less number defined as (1−(S/W)), where S=number of dyad-symmetrical bases and W=window (see FIG. 1B). A perfect palindrome has zero polarity at its central axis of dyad symmetry, and a homogenous stretch of DNA consisting on one strand of only one of the four bases (i.e. AAAAAAAAA . . . A) has a polarity value of one.

The algorithm of a preferred embodiment calculates polarity by comparing the nucleotide sequence of a specified window size (number of base pairs) upstream of the tested potential axis of dyad symmetry, against the nucleotide sequence of an equal size downstream from this axis on the complementary DNA strand (see FIG. 1B). In an alternative preferred embodiment described in greater detail hereinbelow, the nucleotide sequence of a specified window size upstream of the tested potential axis of dyad symmetry is compared against the nucleotide sequence of an equal size downstream from this axis on the same DNA strand. A further feature is that the algorithm may perform this routine for each potential axis of dyad symmetry along the input sequence (see FIGS. 1A and B) and return an ordered list of polarity values for all of the axes tested (see FIG. 1C). A margin, equal in size to the window of symmetry, must be excluded from analysis at each end of the input sequence. This is due to the fact that if an axis is chosen within this margin, the size of the window will exceed the number of bases present on one DNA strand, between the axis and the end of the input sequence. There exists one potential axis of dyad symmetry on each nucleotide base and one between every two consecutive bases (see FIG. 1A).

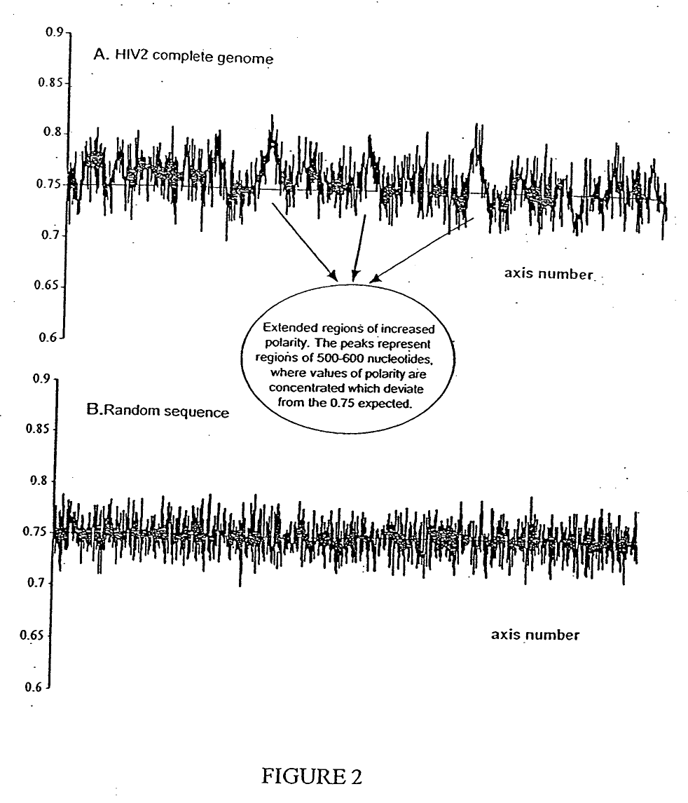

According to further features of the preferred embodiment, once the list of polarity values for all individual potential axes of dyad symmetry in the tested sequence is obtained, its content may be displayed in a graph (see FIG. 1C, and FIG. 2). The graph presents the polarity value at each potential axis of dyad symmetry (or a moving average of groups of axes) along the tested sequence as the y coordinate. The abscissa (x) values of the graph are the axis numbers and can be readily associated with nucleotide positions on the input sequence. The expected polarity value for a random sequence is 0.75, based on both theoretical calculation and experimental data with randomly generated sequence (see FIG. 2B). According to still further features of the preferred embodiment of the present invention, statistical analysis can be performed on the list of polarity values. Statistical analysis can be performed to calculate a probability ratio indicating the deviation of the observed polarity values from the expected. Standard statistical methods which will be familiar to those ordinarily skilled in the art may be used (see Brezinski DP (1975) Nature 253:128-30.) The specific statistical method to be used may be tailored to different configurations of the present invention. For example, variations in base composition in different organisms and in different regions of the genome (must) warrant the use of different statistical evaluations.

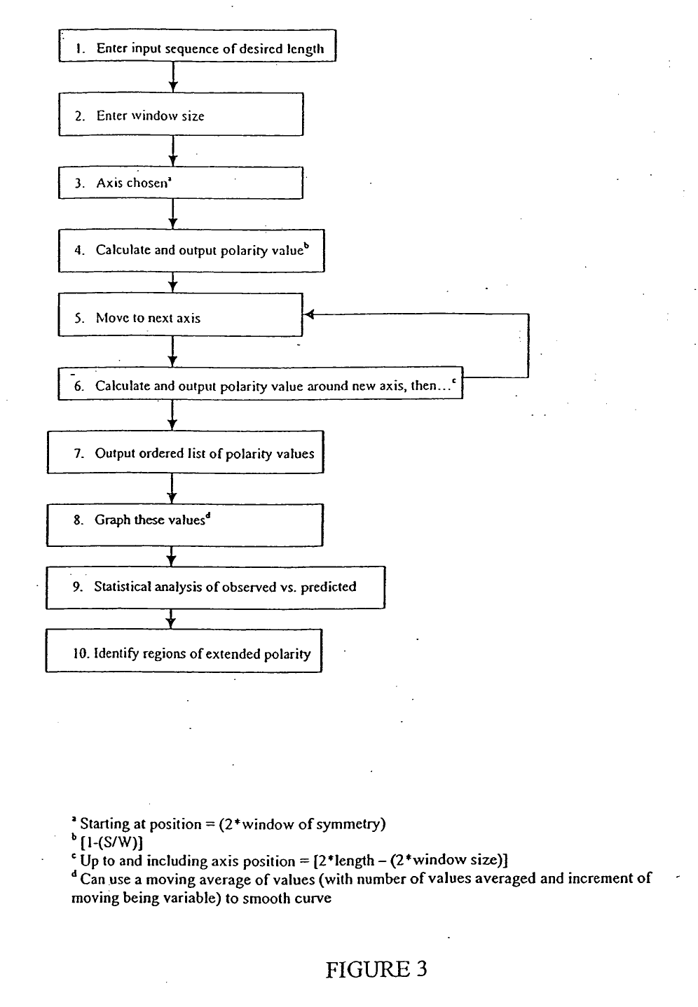

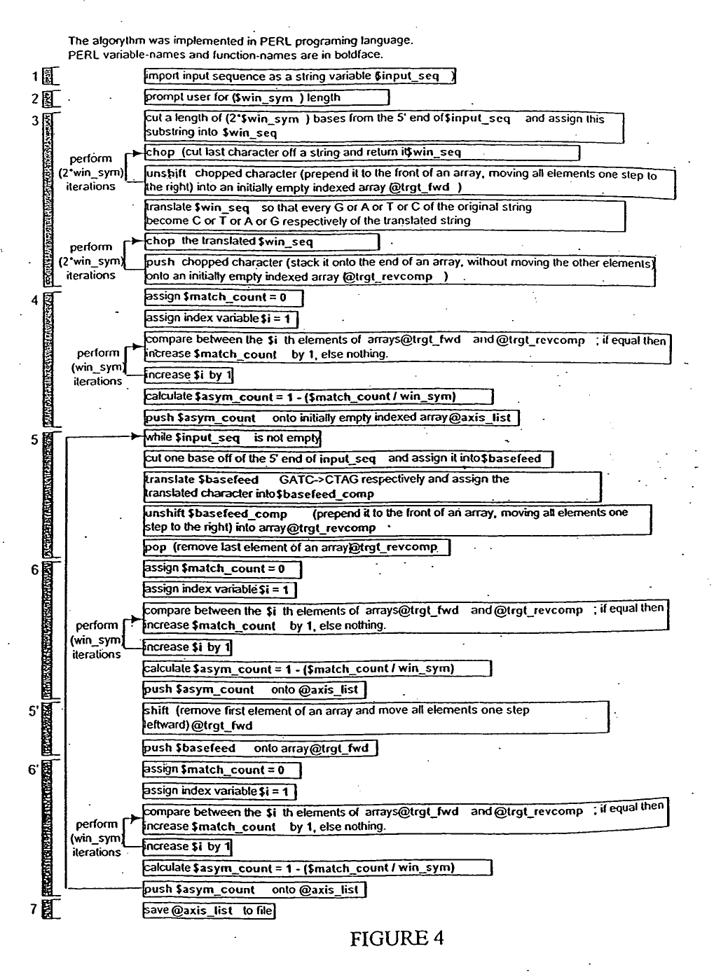

Detailed description of the steps involved in a preferred embodiment of the present invention will be easiest to understand with reference to the numbered steps on the algorithmic flowcharts in FIGS. 3 and 4. FIG. 3 is a flow chart illustrating a specific embodiment of the present invention, with an example of the steps an algorithm for determination of the degree of asymmetry of a defined DNA sequence could take, while the flow chart in FIG. 4 illustrates a further, even more specific example, of a preferred embodiment of the present invention, in the form of an algorithm implemented in PERL programming language. The variable names and functions indicated in bold in FIG. 4 are used by way of example and no details in these examples should be taken as limiting the application of this invention. The first step (1) is for a nucleotide sequence of a single strand of DNA (input sequence, $input_seq) of a desired length to be input. The sequence may be of any length from two bases to 3×10⁹ bases, preferably from 5,000 to 50,000, and most preferably from 10,000 to 20,000. The second step (2) is the input of length of the desired window size ($win_sym). Window size (W as described hereinabove) may be any number from one to a value equal to that of the largest whole integer smaller than one half the length of the DNA sequence, preferably from 20 to 300 and most preferably from 80 to 100. Beginning then at 3, the algorithm starts at the first potential axis of symmetry (axis position=2*the window size) and calculates and outputs a polarity value for that axis (4). In the detailed example of FIG. 4, the input sequence is converted to the two complementary sequence indexed arrays: @trgt_fwd and @trgt_revcomp in the steps indicated as 3 using string $win_seq in the process of these steps. The algorithm tests all the pairs of isometric bases within the window around that axis for identity. In FIG. 4, each base within that window is indexed using variable $i and the number of identical bases (S, as described hereinabove) is counted in variable $match_count. Polarity is calculated using the formula as described hereinabove, polarity=(1−(S/W)). In the algorithm of the example in FIG. 4, polarity is recorded as the variable $asym_count and output to an indexed array, @axis_list. In the next steps 5 and 5′, the algorithm advances to the next potential axis; variables $basefeed and $basefeed_comp are used to advance the axis and sequence in the example of FIG. 4. In steps 6 and 6′, polarity value around the new axis is calculated and output and steps 5/5′ and 6/6′ are repeated iteratively up to and including axis position=2*length of the input sequence−(2*window size). In step 7 the ordered list of polarity values around each potential axis of symmetry is output. Graphical, step 8, and statistical (step 9) analysis can be performed, allowing for identification of extended regions of increased polarity, step 10. For example, it can easily be seen in FIG. 2A that such regions are easily identifiable. Extended regions with decreased polarity as compared to expected are also identified and are also predicted to possess unique charge conductivity properties, namely high resistance.

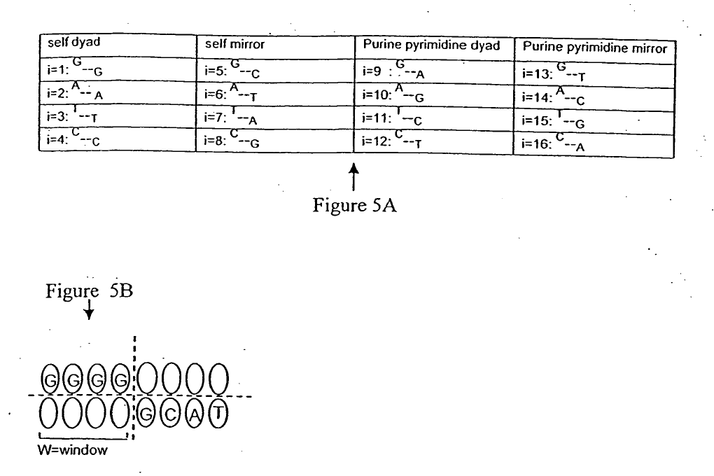

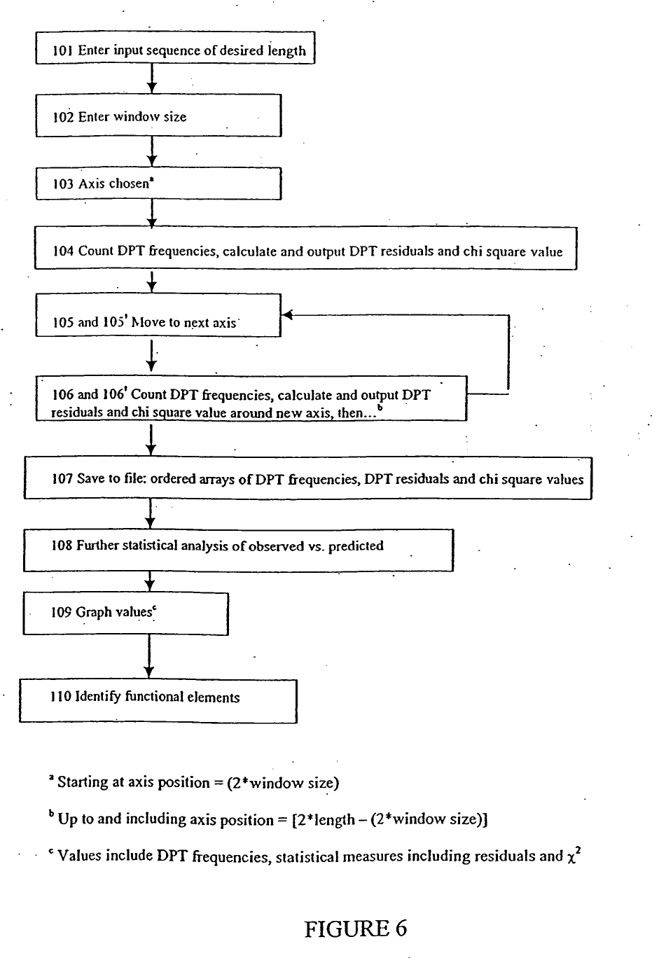

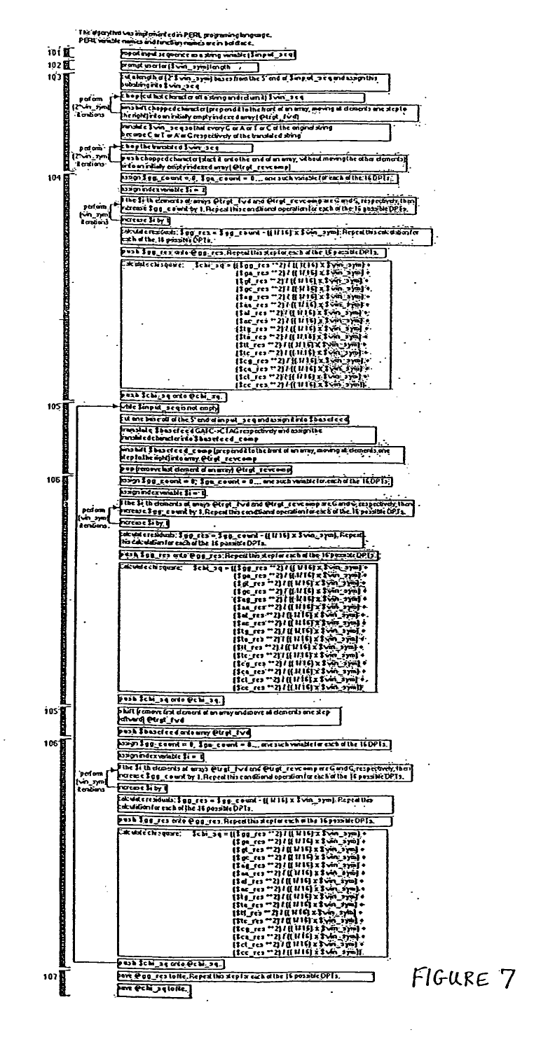

An alternative preferred embodiment performs a more detailed analysis of dyad symmetry. The two bases that are situated at the same position (distance) relative to an axis perpendicular to the major longitudinal axis of a DNA molecule, but located on opposite strands of double stranded DNA, are referred to as a dyad pair. Each dyad pair is one of 16 possible permutations of bases, as illustrated in FIG. 5A. Each of these 16 permutations is referred to as a dyad pair type (DP1). The 16 DPTs can be grouped into four groups: self dyad, self mirror, purine-pyrimidine dyad, and purine-pyrimidine mirror, as illustrated in FIGS. 5A and 5B. FIG. 5B illustrates an 8 base pair fragment of DNA, a potential axis of symmetry at the center of the fragment and a window size of 4. The dyad pairs, from the axis outward, are examples of self dyad (^{G}-_{G}), self mirror (^{G}-_{C}), purine-pyrimidine dyad (^{G}-_{A}), and purine-pyrimidine mirror (^{G}-_{T}) DPTs, respectively. Thus the self mirror group, for example, consists of the dyad pairs: ^{G}-_{C} (as seen in the second dyad pair in FIG. 5B), ^{A}-_{T}, ^{T}-_{A}, and ^{C}-_{G}. In an alternate preferred embodiment of the present invention, illustrated schematically in the flow chart of FIG. 6, at each potential axis of symmetry, rather than calculating symmetry and polarity, the algorithm calculates the frequencies of each of the 16 possible DPTs of the sequence within a fragment of sequence equal to twice the size of a defined window of symmetry, relative to the central axis of that fragment. The sum of the four DPT frequencies in the self dyad group is the same as the symmetry measure (“s”) in the preferred embodiment described hereinabove. The sum of the frequencies of the self mirror, purine-pyrimidine dyad, and purine-pyrimidine mirror groups together is equivalent to the polarity measure (p) in the preferred embodiment described hereinabove. Thus, this preferred embodiment gives finer resolution than the analysis of the preferred embodiment described hereinabove and illustrated in FIGS. 3 and 4. After determining the set of DPT frequencies, and statistical measures (as described hereinbelow), at the first potential axis of symmetry in the input DNA sequence, the algorithm advances to the next potential axis of symmetry, reiterates the calculation of DPT frequencies and associated statistical measures, and moves on until the end of the input sequence is reached. This is done in a manner analogous to that described hereinabove for the preferred embodiment illustrated in FIGS. 3 and 4. An ordered array of DPT frequencies and statistical measures around each potential axis of symmetry is output.

As described hereinabove for the preferred embodiment illustrated in FIGS. 3 and 4, in the preferred embodiment illustrated in FIGS. 6 and 7, statistical and graphical analysis can be performed, allowing for identification of regions predicted to possess unique charge conductivity properties and regions predicted to encode functional elements. To assess the statistical significance of observed deviations from expected DPT frequencies a chi-square (χ²) statistic may be calculated as a non-limiting example. The χ² value for each axis and window is calculated according to the formula:

where i is the DPT as indicated in FIG. 5A, Obᵢ is the observed DPT frequency

$${χ}^{2} = {\sum }_{i = 1}^{16} \frac{{( {Ob}_{i} - Ex )}^{2}}{{Ex}_{i}}$$

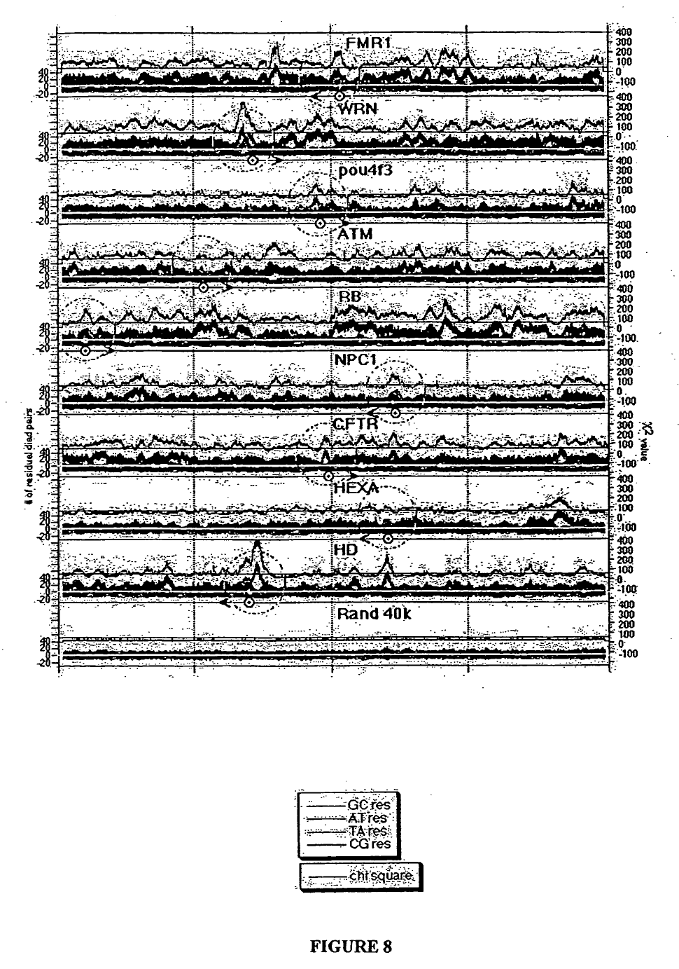

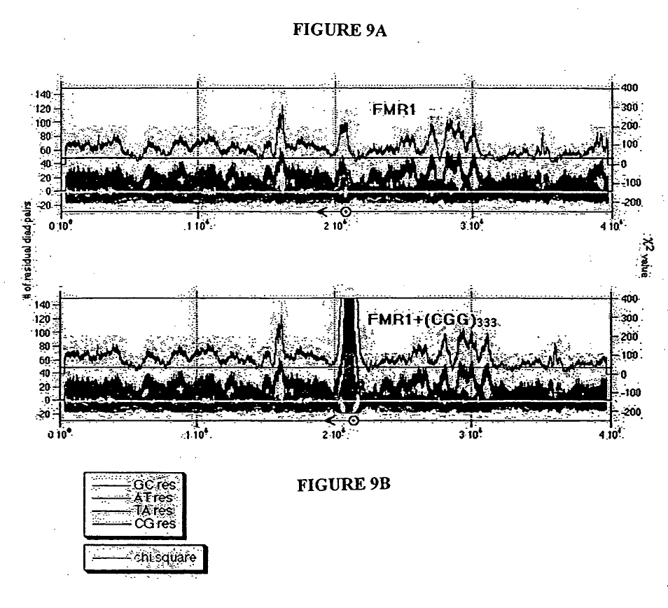

for that DPT and Exᵢ is the expected DPT frequency for that DPT. In calculating the χ² value, expected DPT frequencies are preferably calculated based on a model in which a probability of 1/(4²) is assigned to each DPT. This probability model is based on the assumption of unbiased nucleotide composition, as expected for a random sequence. Thus, Ex=(1/16)×W. In other configurations, the expected frequencies can be based on a model using actual base composition, as counted in each window, or as counted for the entire fragment (two windows on either side of the axis combined), or it can be based on actual base composition, as counted for a particular chromosome, part of a chromosome, or the whole genome of a particular organism. As further non-limiting examples, a residual of the DPT frequency, where Residualᵢ=(Obᵢ−Exᵢ), or a likelihood ratio indicating the deviation of the observed DPT frequencies from the expected also can be calculated. The χ² values, residuals, likelihood ratios and DPT frequencies can be graphically plotted against their axis position in the input sequence. A fragment of computer generated random sequence subjected to the same analysis serves as a negative control and helps to verify that the observations are not an artifact of the analysis and that the χ² value threshold used is appropriate. Examples of such graphical plotting are given in FIGS. 8 and 9, which are discussed in greater detail hereinbelow. As discussed hereinabove, in some configurations, and in some calculations, various dyad pair types may be taken together, such as the 4 major groups as a non-limiting example. In some configurations, some of the statistical analysis of DPT frequency deviation is performed at the time of each set of DPT frequency calculations at each axis rather than following the calculation of all DPT frequencies.

FIG. 6 is a flow chart illustrating a specific preferred embodiment of the present invention, with an example of the steps of an algorithm for determination of the degree of asymmetry of a defined DNA sequence using the calculation of DPT frequencies. The flow chart in FIG. 7 illustrates a further, even more specific example, of a preferred embodiment of the present invention, using the calculation of DPT frequencies, in the form of an algorithm implemented in PERL programming language. The variable names and functions indicated in bold in FIG. 7 are used by way of example and no details in these examples should be taken as limiting the application of this invention. The first step (101) is for a nucleotide sequence of a single strand of DNA (input sequence, $input_seq) of a desired length to be input. The sequence may be of any length from two bases to 3×10⁹ bases, preferably from 5,000 to 50,000, and most preferably from 10,000 to 20,000. The second step (102) is the input of length of the desired window size ($win_sym). Window size (W as described hereinabove) may be any number from one to a value equal to that of the largest whole integer smaller than one half the length of the DNA sequence, preferably from 20 to 300 and most preferably from 80 to 100. Beginning then at 103, the algorithm starts at the first potential axis of symmetry (axis position=2*the window size) and calculates and outputs a set of DPT frequencies for that axis (104). The algorithm then calculates and outputs DPT residuals and one chi-square value per axis as described hereinabove. As for FIG. 4 as described hereinabove, the input sequence is converted to the two complementary sequence indexed arrays: @trgt_fwd and @trgt_revcomp in the steps indicated as 103 using string $win_seq in the process of these steps. In the next steps 105 and 105′, the algorithm advances to the next potential axis; variables $basefeed and $basefeed_comp are used to advance the axis and sequence in the example of FIG. 7. In steps 106 and 106′, DPT frequencies, residuals and chi-square values around the new axis are calculated and output and steps 105/105′ and 106/106′ are repeated iteratively up to and including axis position=2*length of the input sequence−(2*window size). In step 107 the ordered arrays of DPT frequencies, DPT residuals and chi-square values around each potential axis of symmetry are saved to a file. Further statistical, step 108, and graphical (step 109) analysis can be performed, allowing for identification of functional elements, step 110.

Further envisioned as being within the scope of the present invention are alternate configurations wherein the nucleotide sequence of a specified window size upstream of the tested potential axis of dyad symmetry is compared against the nucleotide sequence of an equal size downstream from this axis on the same, rather than the complementary, DNA strand. In the preferred embodiments illustrated in FIGS. 3 and 6, a dyad pair on complementary strands is analyzed in order to determine s, and therefore p, or in order to determine DPT frequencies. Alternatively the sequence of only one single strand can be analyzed, based on the complementary nature of the strands. As a non-limiting example, rather than checking to see if the tvo bases that are situated at the same position relative to a potential axis of symmetry, but located on opposite strands of double stranded DNA, are identical, in this configuration the two bases that are situated at the same position relative to a potential axis of symmetry, but located on the same DNA strand, (referred to as mirror pair), are examined to check whether they are complementary. For example, a ^{G}-_{G} dyad pair and a ^{G}-^{C} mirror pair are the same entity.

Local biases in nucleotide composition can strongly contribute to dyad pair type frequency deviation because the frequencies of dyad pair types are proportional to the frequencies of occurrence of the bases from which they are comprised. For example, a GC rich region of DNA will have higher frequencies of the ^{G}-_{G} and ^{G}-_{C} dyad pair types. Thus, specifically envisioned as being within the scope of the present invention are alternate configurations wherein the frequencies of bases within a given region of DNA sequence of a defined window size on either side of a potential axis is determined. Such a method of determining nucleotide composition frequencies is inferior in accuracy to directly determining DPT frequencies because it neglects the effect of base order and therefore captures less of the available information than the direct DPT frequency analysis. Nucleotide composition frequency analysis can however be used for the same purpose of locating functional elements and evaluating electrical conductivity, in a very similar way to the method described, albeit in a less definitive manner.

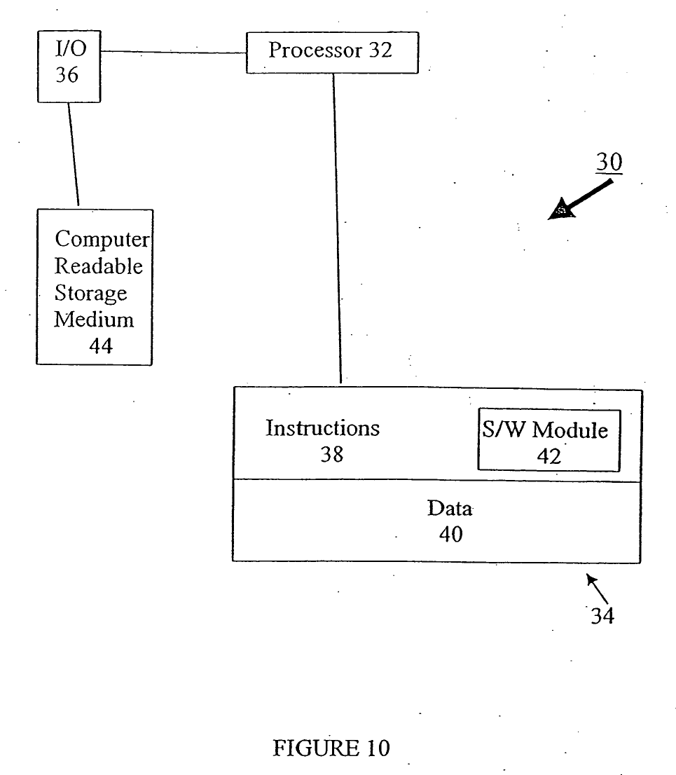

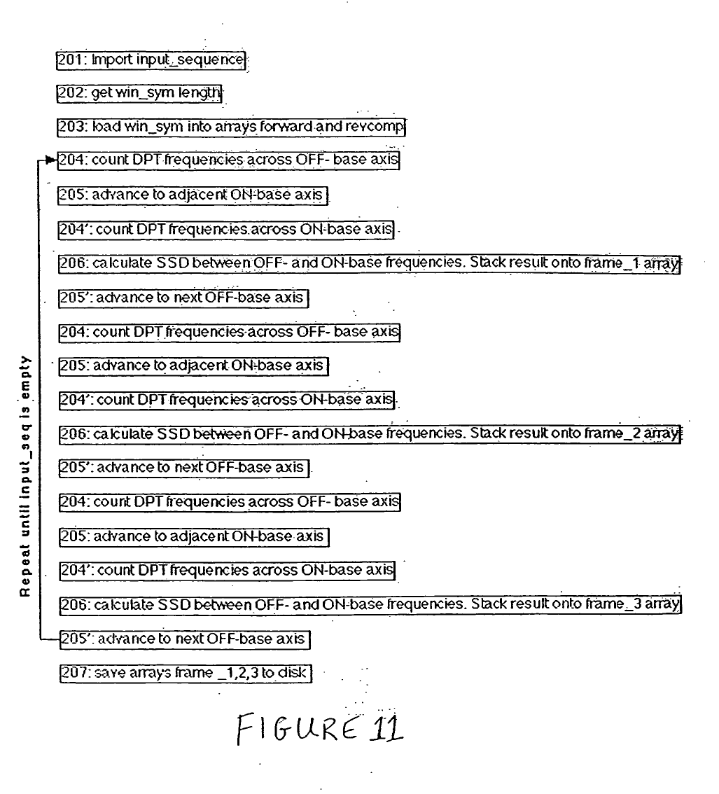

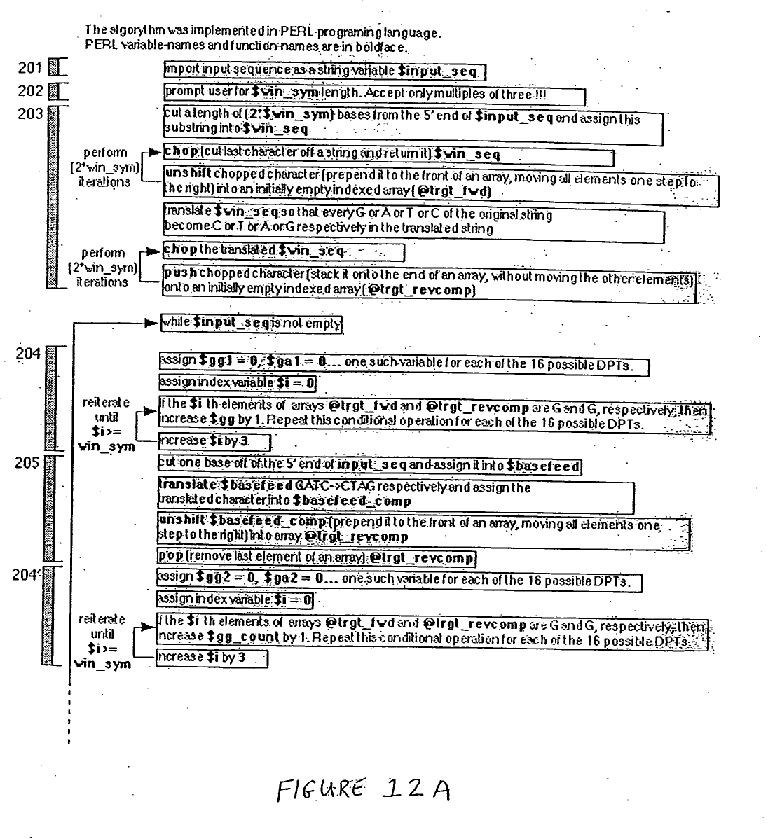

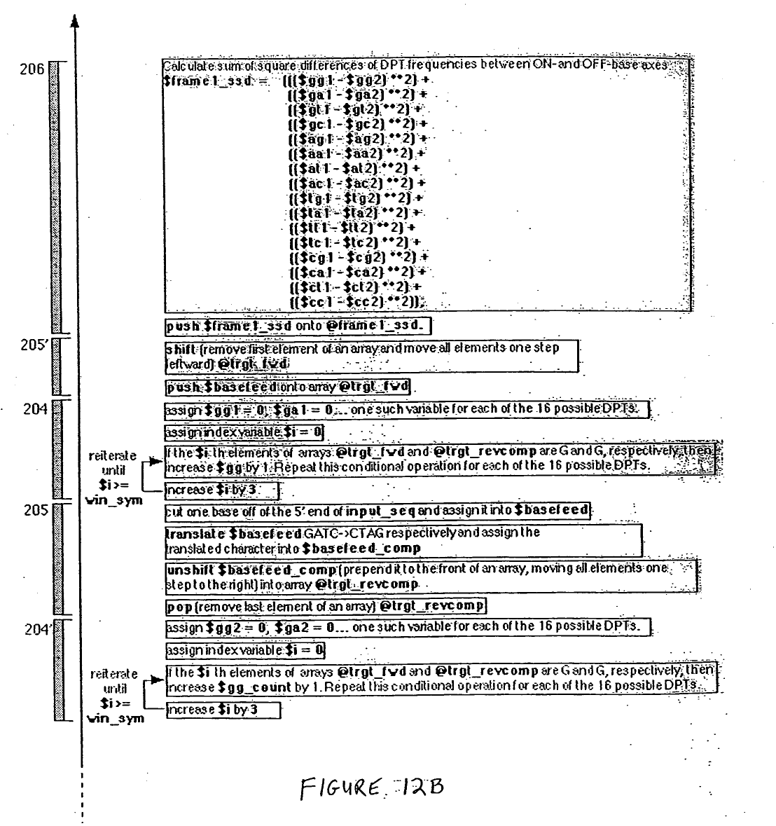

FIG. 10 is a high level block diagram of a system 30 for predicting the electrical conductivity properties and for identifying functional elements in a defined DNA sequence according to the present invention. System 30 includes a processor 32, a random access memory 34 and a set of input/output devices, such as a keyboard, a floppy disk drive, a printer and a video monitor, represented by I/O block 36. Memory 34 includes an instruction storage area 38 and a data storage area 40. Within instruction storage area 38 is a software module 42 including a set of instructions which, when executed by processor 32, enable processor 32 to calculate dyad pair type frequencies, perform statistical analyses and graphical plotting by the method of the present invention.

Using the appropriate input device 36 (typically a floppy disk drive), source code of software module 42, in a suitable high level language, for calculating dyad pair type frequencies, and performing statistical analyses according to the present invention is loaded into instruction storage area 38. The source code of software module 42 is provided on a suitable computer readable storage medium 44, such as a floppy disk or a compact disk. This source code is coded in a suitable high-level language.

Selecting a suitable language for the instructions of software module 32 is easily done by one ordinarily skilled in the art. The language selected should be compatible with the hardware of system 30, including processor 32, and with the operating system of system 30. If a compiled language is selected, a suitable compiler is loaded into instruction storage area 38. Following the instructions of the compiler, processor 32 turns the source code into machine-language instructions, which also are stored in instruction storage area 38 and which also constitute a portion of software module 42. Using the appropriate input device 36, the parameters of the DNA sequence analysis are entered, and are stored in data storage area 40. The results of the analysis are displayed at video monitor 36 or printed on printer 36.

While reducing the present invention to practice, several results pertaining to the identification of functional elements in DNA sequence were obtained. These results clearly show that the method and system of the present invention identifies functional elements such as transcription related functional elements including promoters and enhancers and identifies alterations related to regulatory domain mutations underlying human genetic diseases.

Additional objects, advantages, and novel features of the present invention will become apparent to one ordinarily skilled in the art upon examination of the following examples, which are not intended to be limiting.

Graphs of DPT frequency variation in several human genomic fragments, each containing a disease-associated gene, are presented in FIG. 8. Shown are output plots of dyad pair type frequency analyses of nine human genomic sequence fragments each containing a condition associated gene and one control fragment of computer generated random sequence. Each fragment is 40 kilobases (kb) long. A window of length 300 basepairs (bp) was used in all analyses shown. The start site of the primary transcript of each gene is marked by a yellow circle on the x-axis of the graph to which it corresponds, with an arrow indicating the direction of the gene. The x-axis represent bp positions on the + strand of the GenBank entry. The horizontal yellow line on each graph corresponds to zero on the left Y scale (number of residual dyad pairs); the black line corresponds to 30.6 on the right Y scale, which is the value of the threshold χ² (15 degrees of freedom; α=0.01). At residual values of zero, there is no difference between the observed and expected DPT frequencies; above, there is overexpression of that DPT, and below, there is a supression of that DPT. All axes with χ² values above the 30.6 threshold have, around that axis, DPT frequencies that deviate significantly from the expected. The sequences presented are: FMR1: Fragile-X mental retardation (GenBank accession #L_{—}29074, bp 1-40k analyzed); WRN: Werner Syndrome (GenBank accession #181896, bp 1-40k analyzed); POU4F3: Hearing impairment (GenBank accession #NT_{—}006700, bp 120k-160k analyzed); ATM: Ataxia Telangiectasia (GenBank accession #U82828, bp 1-40k analyzed); RB: Retinoblastoma (GenBank accession #L11910, bp 1-40k analyzed); NPC1: Niemann Pick C1 syndrome (GenBank accession #NT_{—}011044, bp 220k-260k analyzed); CFTR: Cystic Fibrosis (GenBank accession #AC_{—}000111, bp 1-40k analyzed); HEXA: Tay Sachs syndrome (GenBank accession #NT_{—}010303, bp 190k-230k analyzed); and HD: Huntington disease (GenBank accession #88756, bp 1-40k analyzed).

Some important features, common to all the graphs in FIG. 8, are: (a) Significant deviation of DPT frequencies exists in the majority of axes in the sequences presented (all points with χ² values above the α=0.01 threshold line are significant). (b) In all of the sequences shown, the ^{T}-_{A} and ^{A}-_{T} DPTs are over represented and the ^{G}-_{C} and ^{C}-_{G} DPTs are suppressed, and (c) There appears to be a defined pattern of DPT frequency variation, which coincides with the transcription start sites of genes. A dashed line circle is drawn around the DPT frequency variation element, coinciding with the transcription initiation site of each of the genes shown. This DPT variation element consists of a local, steep increase in the frequencies of the ^{G}-_{C} and/or ^{C}-_{G} DPTs, with a concomitant decrease in the frequencies of the ^{T}-_{A} and ^{A}-_{T} PTs. The length of the element is typically 1-2 kb, but is widely variable from gene to gene.

Over 50 genes were analyzed so far, in the overwhelming majority of them a recognizable variant of the element was found to reside on the transcription start site. Although variations in the element are considerable from gene to gene, it appears that the main features of the element are conserved. In addition to the transcription start site element, there are other, distinct, elements distributed in genomes of various organisms including viruses, bacteria and eukaryotes. DPT frequency deviation is indicative of anisotropy in an underlying physical property of the double helix, a property that is related to charge conductivity. Regions of DNA that possess DPT frequency deviation thus are used to identify both regions with altered electrical conductivity as well as functional elements within the DNA sequence.

The present invention further permits identification and understanding of mutations underlying genetic conditions. FIG. 9 shows a DPT frequency analysis of two 40 kb sequences, with a window length of 300 bp. In the lower graph 9B, the scales of the Y axes are identical to those in the upper graph 9A, although the maximal values of chi square in graph 9B far exceed the maximal value on the axis and reach their maximum at >1800. The analysis in graph 9A is of the FMR1 fragment containing bases 235k-275k from the GenBank entry accession #NT_{—}011744. In graph 9B, the DPT analysis is of the “FMR1+(CGG)₃₃₃” fragment which was obtained by inserting a 1000 bp fragment (containing 333 tandem repeats of the (CCG) trinucleotide) into the sequence described for graph 9A, at position 255459 of the GenBank entry #NT_{—}011744. The fragment was inserted at the site of the (CGG) repeat, expansion of which was shown to cause Fragile-X Syndrome. The “FMR1+(CGG)₃₃₃” fragment thus simulates an expanded allele with approximately 350 (CGG) repeats. When affected and non-affected alleles of the gene known to be responsible for Fragile-X syndrome (FMR1) are analyzed and compared using the present invention, a clearly visible difference is found between the transcription initiation site elements of the affected and non-affected alleles. As many genetic conditions may be caused by mutations in regulatory control regions, the system and method of the present invention will not only permit identification of such mutations but will also therefore contribute to a better understanding of the control of gene expression in both normal and pathological situations.

In an alternate preferred embodiment, dyad symmetry analysis of nucleic acid sequence can be used as a tool for assessing the information content of that sequence. DNA is the carrier of genetic information and as such the two major functions of DNA are to store the genetic information and to transfer that genetic information from generation to generation. Information in this context is defined as the set of all instructions and commands necessary for the formation and maintenance of a living organism, which are stored in DNA and RNA. In this definition are included complete sets of such instructions and commands, as stored in complete genomes, as well as all subsets thereof such as these included in viruses, plasmids and artificial clones of recombinant DNA.

The present invention includes a method with a general applicability to nucleic acid systems of information storage, that will detect and measure the information content along a nucleic acid sequence and also assist in decoding that information. A preferred embodiment of such a method and a system for executing it is presented here containing an algorithm which determines the level of dyad symmetry across potential axes of dyad symmetry in the nucleic acid molecule. The ability of this method to predict the electrical conductivity of regions in DNA, as part of a putative mechanism of information storage related to transcription control, was demonstrated hereinabove. In an alternative preferred embodiment the ability of the method and system of the present invention to predict functional elements of transcription was demonstrated hereinabove. In a yet additional preferred embodiment, the ability of the method and system of the present invention to predict protein-coding regions is demonstrated.

In the information storage system known as the ‘Genetic Code’, which provides the transition rules from DNA to protein, the instructions are written in a three-letter code. The specific embodiment described herein takes advantage of this fact to provide a tool for assessing information content related specifically to protein coding. The three letter codons in DNA sequence are concatenated, without spaces. The message is ‘frame specific’. Because of the degeneracy of the genetic code, the first position in each codon is in general the most rigid, the second and third are increasingly more flexible. Based on these rules this alternate preferred embodiment compares DPT frequencies across axes of symmetry which are ON base pairs to DPT frequencies which are BETWEEN base pairs. For the purposes of this specification and the accompanying claims, the terms “between axis,” “between base-pair axis,” “off base-pair axis,” “off base axis,” and “off axis” shall be interpreted as meaning the same thing. The analysis is designed to be frame specific, and compares only the first position in each codon to its potentially dyad symmetrical counterpart. In this way a high level of sensitivity is achieved (separation of the significant patterns from the background noise of stochastic fluctuations), specifically for protein coding regions.

The preferred embodiment of a method and system for predicting and identifying protein coding regions in a defined nucleic acid sequence using an analysis of dyad symmetry, specifically using an analysis of DPT frequencies, according to the present invention is directly analogous to the preferred embodiment described hereinabove for deter ining functional elements and as illustrated in figures in FIGS. 5-7 and 10, except as noted hereinbelow.

FIG. 11 is a simplified flow chart that illustrates steps 201-207 which are analogous to steps 101-107 described hereinabove and in FIGS. 6 and 7 except as detailed here. Steps 201 and 203 are identical to steps 101 and 103 respectively. Step 202 is different from 102 only in the fact that only multiples of three are: accepted for win_sym length, to fit codon size. Step 204 is a count of DPT frequencies with an increment step of 3, across an OFF-base pair axis. Step 204′ is the same as step 204 except that step 204′ is across an ON-base pair axis. Steps 205 and 205′ are identical to steps 105 and 105′ and are used to shift the axis from an OFF-base axis to an ON-base axis and vice versa, respectively. Step 206 is the calculation step in this preferred embodiment; in step 206 the sum of square differences (SSD) is calculated, between DPT frequencies across an OFF-base axis and the ON-base axis adjacent to it, according to the formula:

$$SSD = {\sum }_{i = 1}^{16} {( {BB}_{i} - {OB}_{i} )}^{2}$$

BB=(Between_base_axis_frequency)ᵢ

OB=(On_base_axis_frequency)ᵢ

i=index_in_DPT_table

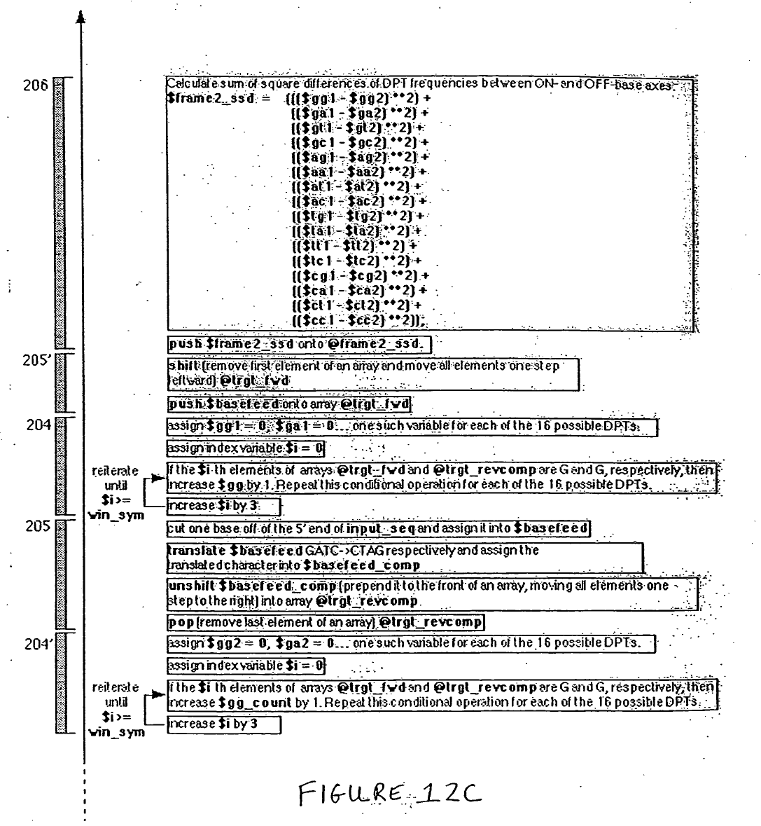

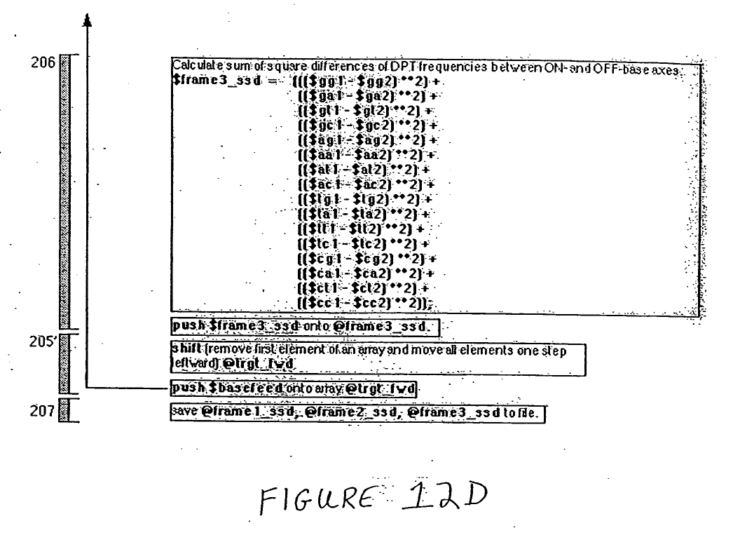

The result is stacked onto an array; there is one such array for each frame. In step 207 the three result arrays are saved to file. There are three repetitions of a basic block (204, 205, 204′, 206, 205′)₃, one such repetition for each reading frame. This process is reiterated until the end of input_sequence is reached. More details are shown in FIG. 12, parts A-D, where part B follows the last step illustrated in part A, part C follows the last step in part B, and part D follows the last step in part C. Following step 207 further statistical (208) and graphical (209) analysis can be performed, allowing for the identification of protein coding regions (210) analogous to steps 108-110, but not illustrated.

The preferred embodiment of a method according to the present invention for identifying protein coding regions in a defined nucleic acid sequence using an analysis of dyad symmetry, thus calculates DPT frequencies, in the same manner as the embodiments described hereinabove, and it compares DPT frequencies of windows centered around between base axes to those of the adjacent axes located on base. The method of the preferred embodiment moves 5′ to 3′ on the input sequence and calculates between base axis DPT frequencies first. Alternate configurations in which the movement is from 3′ to 5′ and in which on base axis DPT frequencies are calculated first are within the scope of the present invention. Further, as for the embodiments described hereinabove, in certain configurations the analysis may be performed with a dyad pair on the same nucleic acid strand or in alternative configurations on a dyad pair where the two bases are situated at the same position relative to a potential axis of symmetry but located on opposite (reverse complementary) strands of nucleic acid.

Further, specifically envisioned as being within the scope of the present invention are alternate configurations wherein the frequencies of bases within a given region of DNA sequence of a defined window size on either side of a potential axis is determined. Such a method of determining nucleotide composition frequencies is inferior in accuracy to directly determining DPT frequencies because it neglects the effect of base order and therefore captures less of the available information than the direct DPT frequency analysis. Nucleotide composition frequency analysis can however be used for the same purpose of locating protein encoding regions of nucleic acid sequences, in a very similar way to the method described, albeit in a less definitive manner.

More advanced embodiments are executable which further accommodate variability in factors which influence the outcome of the analysis, such as exon length, frame shifting errors in the database and pseudogenes.

Further, a system for predicting and identifying protein coding regions in a defined nucleic acid sequence using an analysis of dyad symmetry, specifically using an analysis of DPT frequencies, is analogous to the system illustrated in FIG. 10. In this embodiment, software module 42 includes a set of instructions which, when executed by processor 32, enable processor 32 to calculate the DPT frequencies and sum of square differences, and perform statistical analyses and graphical plotting according to the method of the present invention.

This embodiment has the potential to serve as a tool for predicting protein coding information content, based on sequence analysis alone, thus saving on expression library screening and other costly laboratory procedures. It may also prove useful in gene discovery as some RNA transcripts expressed in low abundance or in extremely narrow time windows in development, are virtually absent from expression libraries and can only be inferred from sequence. This embodiment is distinct from any previously published algorithm, as prior art methods are all based on pattern searches of specific words and their association with functional elements and none are based on systematic comparison of the 5′→3′ sequences of a sliding window, such as this present invention.

While reducing the present invention to practice, several results pertaining to the identification of protein encoding regions (exons) in nucleic acid sequence were obtained. These results clearly show that the method and system of the present invention identifies protein-encoding regions in a defined nucleic acid sequence.

Additional objects, advantages, and novel features of the present invention will become apparent to one ordinarily skilled in the art upon further examination of the following additional examples, which are not intended to be limiting.

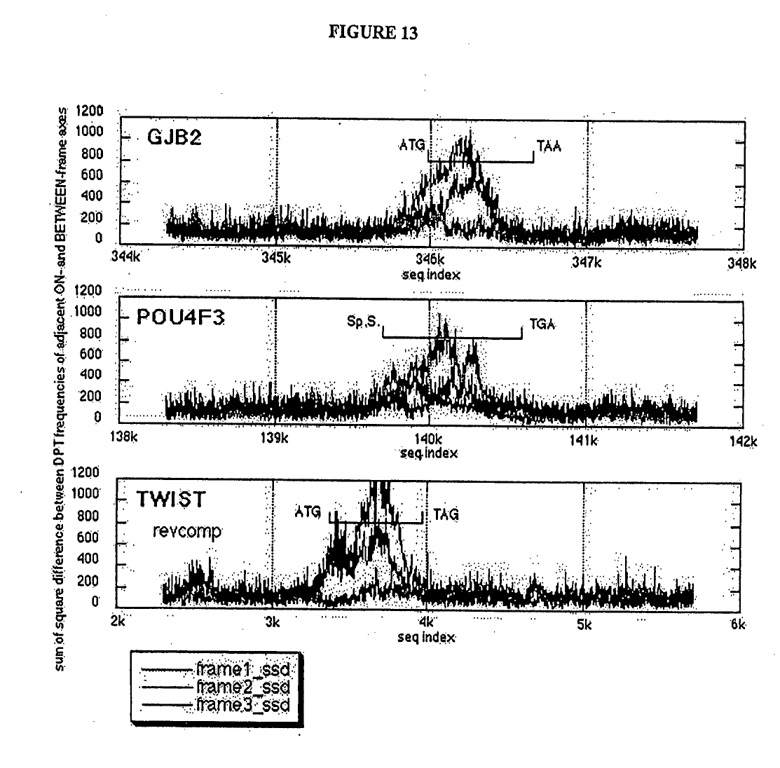

FIG. 13 illustrates a graphical analysis of the output of DPT frequency analyses of genomic sequences from three human genes. Panel a shows the analysis of the GJB2 gene (accession #NT_{—}024521, bases 344k-348k.). A fragment spanning the second exon of the gene is shown, containing the entire coding sequence of the gene. Panel b shows the analysis of the POU4F3 gene (accession #NT_{—}006700, bases 138k-142k.). A fragment spanning the entire length of the gene is shown. Panel c shows the analysis of the TWIST gene (accession #Y10871 reverse complement, bases 2k-6k). A fragment spanning the entire length of the gene is shown. A 4000 bp fragment is shown from each gene. Analyses were performed with win_sym=300. In the graphs, the Sum of Square Difference between DPT frequencies of adjacent ON- and BETWEEN-base axes is plotted on the Y-axis, with the base positions being tracked along the X-axis. Each of the 3 lines in each graph corresponds to one potential reading frame. The graph reveals an increase in the SSD (between ON-base pair and BETWEEN-base pair axes) associated with coding sequence. In FIG. 13, ATG+1 denotes the first codon of the putative polypeptide and TAA, TAG, TGA the stop codons with Sp.S denoting a splice site.

Thus protein-encoding regions of the nucleic acid sequences can be identified. The ability of the embodiments of the present invention to be used for the identification of functional elements and coding sequences demonstrates that regions that are known to contain information may be detected by DPT analysis.

The method and system for predicting and identifying information containing regions (including protein coding regions) in a defined nucleic acid sequence using an analysis of dyad symmetry, specifically using an analysis of DPT frequencies, according to the present invention have a number of uses. The possible uses are to decipher any form of coded genetic information stored in the DNA molecule including instructions to the transcription apparatus, translation apparatus, DNA packaging and architecture apparatus (nucleosomes etc.) and any form of information not yet even hypothesized which may be contained in the DNA molecule.

The overwhelming majority of genes are discovered through expression libraries. Dyad symmetry analysis detects coding sequence from genomic data without the use of expression data. It will therefore assist in discovery and characterization of new genes that escape detection because of scarce expression. This will also reduce the cost of gene discovery.

The methods and systems according to the present invention utilize a logic different from existing prediction algorithms, which are generally based on sophisticated versions of homology and pattern searches, and therefore the methods and systems of the present invention will help to reveal new genes which do not share homology with pre-discovered genes. The methods and systems according to the present invention can be used to correct frameshift errors in the databases because the method is extremely sensitive to frame. They will help to find splice variations in known genes and verify the integrity of their putative polypeptide products. The methods and systems according to the present invention can be used to verify the integrity of putative polypeptide sequences derived from DNA sequence (which are well known to contain mistaken annotation). This will thus help in reducing the risk of error in protein sequences used for advanced biochemical and structural analyses. The methods and systems according to the present invention can be used to conduct evolutionary surveys, because the level of nucleic acid sequence asymmetry is proportional to the level of specialization of the message (like a more advanced language).

The methods and systems according to the present invention will help in devising better diagnostic tools for genetic diseases, by locating regions coding for information involved in the control of gene expression of disease causing genes. Further, they will help in locating and decoding information contained in DNA which has not yet been decoded.

Although the invention has been described in conjunction with specific embodiments thereof, it is evident that many alternatives, modifications and variations will be apparent to those skilled in the art Accordingly, it is intended to embrace all such alternatives, modifications and variations that fall within the spirit and broad scope of the appended claims.

What is claimed is:

1. A method for determining a measure of electrical conductivity of a defined DNA sequence, the method comprising the step of calculating the degree of asymmetry of the defined DNA sequence.

2. The method of claim 1, wherein said step of calculating said degree of asymmetry is accomplished by calculating at least one polarity value around at least one potential axis of dyad symmetry in the DNA sequence, wherein said polarity value is a unit-less number defined as (1−[S/W]), where S represents a number of dyad-syrnmetrical bases and W represents a window size.

3. The method of claim 2, wherein said at least one polarity value is an ordered series of said polarity values iteratively calculated for each potential axis in the DNA sequence.

4. The method of claim 3, wherein said series of polarity values is plotted graphically, whereby extended regions in the DNA sequence that possess values of polarity which deviate from expected polarity values of a random sequence may be identified.

5. The method of claim 3, wherein said series of polarity values is subjected to statistical analysis, whereby extended regions in the DNA sequence that possess values of polarity which deviate from expected polarity values of a random sequence may be identified.

6. The method of claim 2, wherein the DNA sequence length is in the range of 2 bases to 3×10⁹ bases.

7. The method of claim 2, wherein said window size is an independent variable, with values ranging from 1 to a value equal to that of the largest whole integer smaller than one half the length of the DNA sequence, said variable to be designated prior to calculation.

8. A system for determining a measure of electrical conductivity of a defined DNA sequence, the system comprising, a computer containing within a memory device thereof, an algorithn, said algorithm capable of calculating the degree of asymmetry of the defined DNA sequence.

9. The system of claim 8, wherein said calculation of degree of asymmetry is determined by calculating at least one polarity value around at least one potential axis of dyad symmetry in the DNA sequence, wherein said polarity value is a unitless number defined as (1−[S/W]), where S represents a number of dyad-symmetrical bases and W represents a window size.

10. The system of claim 9, wherein said at least one polarity value is an ordered series of said polarity values iteratively calculated for each potential axis in the DNA sequence.

11. The system of claim 10, wherein said series of polarity values is plotted graphically, whereby extended regions in the DNA sequence that possess values of polarity which deviate from expected polarity values of a random sequence may be identified.

12. The system of claim 10, wherein said series of polarity values is subjected to statistical analysis, whereby extended regions in the DNA sequence that possess values of polarity which deviate from expected polarity values of a random sequence may be identified.

13. The system of claim 9, wherein the DNA sequence length is in the range of 2 bases and 3×10⁹ bases.

14. The system of claim 9, wherein window size is an independent variable, with values ranging from 1 to a value equal to that of the largest whole integer smaller than one half the length of the DNA sequence, said variable to be input into said system by an operator thereof.

15. A method for the evaluation of electrical conductivity of a defined DNA sequence comprising the step of providing instructions on a computer readable medium for a calculation of the degree of asymmetry of the defined DNA sequence.

16. The method of claim 15, wherein said calculation of degree of asymmetry is accomplished by calculating at least one polarity value around at least one potential axis of dyad symmetry in the DNA sequence, wherein said polarity value is a unit-less number defined as (1−[S/W]), where S represents a number of dyad-symmetrical bases and W represents a window size.

17. The method of claim 16, wherein said at least one polarity value is an ordered series of said polarity values iteratively calculated for each potential axis in the DNA sequence.

18. The method of claim 17, wherein said series of polarity values is plotted graphically, whereby extended regions in the DNA sequence that possess values of polarity which deviate from expected polarity values of a random sequence may be identified.

19. The method of claim 17, wherein said series of polarity values is subjected to statistical analysis, whereby extended regions in the DNA sequence that possess values of polarity which deviate from expected polarity values of a random sequence may be identified.

20. The method of claim 16, wherein the DNA sequence length is in the range of 2 bases to 3×10⁹ bases.

21. The method of claim 16, wherein said window size is an independent variable, with values ranging from 1 to a value equal to that of the largest whole integer smaller than one half the length of the DNA sequence, said variable to be designated prior to calculation.

22. A method for identifying functional elements in a DNA sequence, the method comprising the steps of:
(a) calculating at least one set of dyad pair type frequencies within a portion of the DNA sequence equal in size to two times a window size, around at least one potential axis of dyad symmetry in the DNA sequence, and
(b) based on said at least one set of dyad pair type frequencies, identifying regions of the DNA sequence containing the functional elements.

23. The method of claim 22, wherein at least one of said at least one set of dyad pair type frequencies is an ordered array of said dyad pair frequencies.

24. The method of claim 22, wherein said calculating is effected iteratively for each said at least one potential axis of dyad symmetry in the DNA sequence.

25. The method of claim 22, wherein said step of identifying regions of the DNA sequence containing the functional elements is effected by steps including subjecting said at least one set of dyad pair type frequencies to statistical analysis, whereby at least one region in the DNA sequence is identified that possesses at least one statistical value that indicates that an observed said at least one set of dyad pair type frequencies deviates from an expected said at least one set of dyad pair type frequencies.

26. The method of claim 25, where said at least one statistical value is chosen from the group consisting of residuals of said dyad pair type frequencies, chi-square values, and likelihood ratios.

27. The method of claim 25, wherein said statistical analysis includes plotting said statistical values.

28. The method of claim 22, wherein said window size is an independent variable, having a value of at least 1 and at most one half a length of the DNA sequence.

29. The method of claim 22, wherein said calculating of said at least one set of dyad pair type frequencies is performed on at least one dyad pair wherein both nucleotides of said at least one dyad pair are located on a single strand of the DNA sequence.

30. The method of claim 22, wherein said calculating of said at least one set of dyad pair type frequencies is performed on at least one dyad pair wherein both nucleotides of said at least one dyad pair are located on complementary strands of the DNA sequence.

31. A method of identifying transcription related functional elements, comprising the method of claim 22.

32. A system for identifying functional elements in a DNA sequence, the system comprising:
(a) a software module including a plurality of instructions for calculating at least one set of dyad pair type frequencies within a portion of the DNA sequence equal in size to two times a window size, around at least one potential axis of dyad symmetry in the DNA sequence,
(b) a memory for storing said instructions, and,
(c) a processor for executing said instructions.

33. A computer readable storage medium having computer readable code embodied on said computer readable storage medium, the computer readable code for identifying functional elements in a DNA sequence, the computer readable code comprising: program code including a plurality of instructions for calculating at least one set of dyad pair type frequencies within a portion of the DNA sequence equal in size to two times a window size, around at least one potential axis of dyad symmetry in the DNA sequence.

34. A method for determining electrical conductivity properties of a DNA sequence, the method comprising the steps of:
(a) calculating at least one set of dyad pair type frequencies within a portion of the DNA sequence equal in size to two times a window size, around at least one potential axis of dyad symmetry in the DNA sequence, and,
(b) based on said at least one set of dyad pair type frequencies, determining the electrical conductivity properties of the DNA sequence.

35. A system for determining electrical conductivity properties of a defined DNA sequence, the system comprising:
(a) a software module including a plurality of instructions for calculating at least one set of dyad pair type frequencies within a portion of the DNA sequence equal in size to two times a window size, around at least one potential axis of dyad symmetry in the DNA sequence,
(b) a memory for storing said instructions, and,
(c) a processor for executing said instructions.

36. A computer readable storage medium having computer readable code embodied on said computer readable storage medium, the computer readable code for determining electrical conductivity properties of a defined DNA sequence, the computer readable code comprising: program code including a plurality of instructions for calculating at least one set of dyad pair type frequencies within a portion of the DNA sequence equal in size to two times a window size, around at least one potential axis of dyad symmetry in the DNA sequence.

37. A method for identifying protein coding regions in a nucleic acid sequence, the method comprising the steps of:
(a) calculating at least one set of dyad pair type frequencies within a portion of the nucleic acid sequence equal in size to two times a window size, around at least one potential axis of dyad symmetry in the nucleic acid sequence, and
(b) based on said at least one set of dyad pair type frequencies, identifying the protein coding regions contained within the nucleic acid sequence.

38. The method of claim 37, wherein said step of identifying the protein coding regions contained within the nucleic acid sequence is effected by steps including comparing two of said at least one set of dyad pair type frequencies from around two adjacent of said at least one potential axis of dyad symmetry in the nucleic acid sequence.

39. The method of claim 38, wherein said comparing is effected by steps including calculating a sum of squares differences between said at least one set of dyad pair type frequencies from around two adjacent of said at least one potential axis of dyad symmetry in the nucleic acid sequence.

40. The method of step 39, wherein one potential axis of dyad symmetry of said two adjacent of said at least one potential axis of dyad symmetry in the nucleic acid sequence is located on a base of the nucleic acid sequence and the other potential axis of dyad symmetry is located off said base.

41. The method of claim 38, wherein said comparing is effected iteratively for each said at least one potential axis of dyad symmetry in the nucleic acid sequence.

42. The method of claim 37, wherein said window size is an independent variable, having a value of at least 1 and at most one half a length of the nucleic acid sequence.

43. The method of claim 37, wherein said calculating of said at least one set of dyad pair type frequencies is performed on at least one dyad pair wherein both nucleotides of said at least one dyad pair are located on a single strand of the nucleic acid sequence.

44. The method of claim 37, wherein said calculating of said at least one set of dyad pair type frequencies is performed on at least one dyad pair wherein both nucleotides of said at least one dyad pair are located on complementary strands of the nucleic acid sequence.

45. A system for identifying protein coding regions in a nucleic acid sequence, the system comprising:
(a) a software module including a plurality of instructions for calculating at least one set of dyad pair type frequencies within a portion of the nucleic acid sequence equal in size to two times a window size, around at least one potential axis of dyad symmetry in the nucleic acid sequence,
(b) a memory for storing said instructions, and,
(c) a processor for executing said instructions.

46. A computer readable storage medium having computer readable code embodied on said computer readable storage medium, the computer readable code for identifying protein coding regions in a nucleic acid sequence, the computer readable code comprising: program code including a plurality of instructions for calculating at least one set of dyad pair type frequencies within a portion of the nucleic acid sequence equal in size to two times a window size, around at least one potential axis of dyad symmetry in the nucleic acid sequence.

47. A method for identifying information containing regions in a nucleic acid sequence, the method comprising the steps of:
(a) calculating a degree of asymmetry of the nucleic acid sequence within a portion of the nucleic acid sequence, around at least one potential axis of dyad symmetry in the nucleic acid sequence, and
(b) based on said degree of asymmetry, identifying the information containing regions contained within the nucleic acid sequence.

48. The method of claim 47, wherein said calculating said degree of asymmetry is effected by steps including calculating at least one set of dyad pair type frequencies within said portion of the nucleic acid, around said at least one potential axis of dyad symmetry in the nucleic acid sequence.

49. The method of claim 47, wherein said calculating said degree of asymmetry is effected iteratively for each said at least one potential axis of dyad symmetry in the nucleic acid sequence.

50. The method of claim 47, wherein said portion of the nucleic acid sequence is equal in size to two times a window size.

51. The method of claim 50, wherein said window size is an independent variable, having a value of at least 1 and at most one half a length of the nucleic acid sequence.

52. The method of claim 48, wherein said calculating of said at least one set of dyad pair type frequencies is performed on at least one dyad pair wherein both nucleotides of said at least one dyad pair are located on a single strand of the nucleic sequence.

53. The method of claim 48, wherein said calculating of said at least one set of dyad pair type frequencies is performed on at least one dyad pair wherein both nucleotides of said at least one dyad pair are located on complementary strands of the nucleic acid sequence.

54. A system for identifying information containing regions in a nucleic acid sequence, the system comprising:
(a) a software module including a plurality of instructions for calculating a degree of asymmetry of the nucleic acid sequence within a portion of the nucleic acid sequence, around at least one potential axis of dyad symmetry in the nucleic acid sequence,
(b) a memory for storing said instructions, and,
(c) a processor for executing said instructions.

55. The system of claim 54, wherein said calculating said degree of asymrnetry is effected by steps including calculating at least one set of dyad pair type frequencies within said portion of the nucleic acid, around said at least one potential axis of dyad symmetry in the nucleic acid sequence.

56. A computer readable storage medium having computer readable code embodied on said computer readable storage medium, the computer readable code for identifying information containing regions in a nucleic acid sequence, the computer readable code comprising: program code including a plurality of instructions for calculating a degree of asymmetry of the nucleic acid sequence within a portion of the nucleic acid sequence, around at least one potential axis of dyad symmetry in the nucleic acid sequence.

57. The computer readable storage medium of claim 56, wherein said calculating said degree of asymmetry is effected by steps including calculating at least one set of dyad pair type frequencies within said portion of the nucleic acid, around said at least one potential axis of dyad symmetry in the nucleic acid sequence.


SEQUENCE LISTING (1 sequence)

SEQ ID NO: 1 - DNA, 16 bp, Artificial
  THEORETICAL SEQUENCE
tagacaatctgtcgtc